SEQUENCE LISTING (22 sequences)

SEQ ID NO: 1 - DNA, 27 bp, Artificial Sequence
  Primer Sequence 1; Chemically synthesized;
tgttcttgtgcaaaggcaatgctaccg

SEQ ID NO: 2 - DNA, 27 bp, Artificial Sequence
  Primer Sequence 2; Chemically synthesized;
tcttaccctgagttcagttccgtctgc

SEQ ID NO: 3 - DNA, 27 bp, Artificial Sequence
  Primer Sequence 3; Chemically synthesized;
gcccctagcacccgacaagctgagtgt

SEQ ID NO: 4 - DNA, 26 bp, Artificial Sequence
  Primer Sequence 4; Chemically synthesized;
ccaggcccatgaccagatcagcacag

SEQ ID NO: 5 - DNA, 27 bp, Artificial Sequence
  Primer Sequence 5; Chemically synthesized;
aagcgtcgctactcctcccccaagagc

SEQ ID NO: 6 - DNA, 27 bp, Artificial Sequence
  Primer Sequence 6; Chemically synthesized;
gtcacacacagcacgtccaccgaggtc

SEQ ID NO: 7 - DNA, 27 bp, Artificial Sequence
  Primer Sequence 7; Chemically synthesized;
tgccagccaattcaagcccagtccttt

SEQ ID NO: 8 - DNA, 31 bp, Artificial Sequence
  Primer Sequence 8; Chemically synthesized;
acacaacctggaagacaaactacaagagcaa

SEQ ID NO: 9 - DNA, 33 bp, Artificial Sequence
  Primer Sequence 9; Chemically synthesized;
gaaggaaataaattcacagtcaaagaatcaagc

SEQ ID NO: 10 - DNA, 30 bp, Artificial Sequence
  Primer Sequence 10; Chemically synthesized;
aacggcagcgccttcttgctggcacccaat

SEQ ID NO: 11 - DNA, 30 bp, Artificial Sequence
  Primer Sequence 11; Chemically synthesized;
agccatgcgccggaccacgacgtcacgcag

SEQ ID NO: 12 - DNA, 34 bp, Artificial Sequence
  Primer Sequence 12; Chemically synthesized;
gggaggcaacctgctggtcatcgtggccatcgcc

SEQ ID NO: 13 - DNA, 31 bp, Artificial Sequence
  Primer Sequence 13; Chemically synthesized;
gacagtgtatcagtgaaggaatcgctttctg

SEQ ID NO: 14 - DNA, 601 bp, Homo sapiens
  allele (301)..(301): rs1799883 (FABP2); nucleotide indicated by r at position 301 may be either G or A
aagttatggaaaaacaactttaatcagttctcttgatcggattgaacctgaacttctgtagaagcaatctgaatgttcttgtgcaaaggcaatgctaccgagttttcttcccaccctcaaaataaacaaacaaaacataacttggaaaaataaacacttcctatgggatttgactttattttctccattgtcttaccttttacaggtgttaatatagtgaaaaggaagcttgcagctcatgacaatttgaagctgacaattacacaagaaggaaataaattcacagtcaaagaatcaagcrcttttcgaaacattgaagttgtttttgaacttggtgtcacctttaattacaatctagcagacggaactgaactcagggtaagaatttttttttttatgagcaatgcattcttgatttttctacccaatattaaaatgatttctgctctatttcattggatggtttaattaatgcaggtctccttcactaactgaagaagccaatgaagtttgtctacattatatattgcacaaattggcaggatatttaaatatgtttttatttttatacgcatctgtgaagaatctgaattgaacagta

SEQ ID NO: 15 - DNA, 801 bp, Homo sapiens
  allele (401)..(401): rs1801282 (PPARG); nucleotide indicated by s at position 401 may be either C or G
tctgctctgataattctaaatacagtacagttcacgcccctcacaagacactgaacatgtgggtcaccggcgagacagtgtggcaatattttccctgtaatgtaccaagtcttgccaaagcagtgaacattatgacacaactttttgtcacagctggctcctaataggacagtgccagccaattcaagcccagtcctttctgtgtttattcccatctctcccaaatatttggaaactgatgtcttgactcatgggtgtattcacaaattctgttacttcaagtctttttcttttaacggattgatcttttgctagatagagacaaaatatcagtgtgaattacagcaaacccctattccatgctgttatgggtgaaactctgggagattctcctattgacscagaaagcgattccttcactgatacactgtctgcaaacatatcacaaggtaaagttccttccagatacggctattggggacgtgggggcatttatgtaagggtaaaattgctcttgtagtttgtcttccaggttgtgtttgttttaatactatcatgtgtacactccagtattttaatgcttagctcgttgctatcgcgttcatttaaaaacatgttcagaaccttaaaaaaggaaacctaacctaatctattttatctctgtgcatggctcccatttcctgaattttaagcattaaaggtatagttatatccaaaaacaatcctgttcatttttatttcctgagtttgcatagatttcccaagaatacataagggctttttagacttgaagggtcactt

SEQ ID NO: 16 - DNA, 511 bp, Homo sapiens
  allele (256)..(256): rs4994 (ADRB3); nucleotide indicated by y at position 256 may be either T or C
tgagccaggtgatttgggagaccccctccttccttctttccctaccgccccacgcgcgacccggggatggctccgtggcctcacgagaacagctctcttgccccatggccggacctccccaccctggcgcccaataccgccaacaccagtgggctgccaggggttccgtgggaggcggccctagccggggccctgctggcgctggcggtgctggccaccgtgggaggcaacctgctggtcatcgtggccatcgccyggactccgagactccagaccatgaccaacgtgttcgtgacttcgctggccgcagccgacctggtgatgggactcctggtggtgccgccggcggccaccttggcgctgactggccactggccgttgggcgccactggctgcgagctgtggacctcggtggacgtgctgtgtgtgaccgccagcatcgaaaccctgtgcgccctggccgtggaccgctacctggctgtgaccaacccgctgcgttacggcgcactgg

SEQ ID NO: 17 - DNA, 558 bp, Homo sapiens
  allele (303)..(303): rs1042713 (ADRB2); nucleotide indicated by r at position 303 may be either G or A
ctagtctgcgcacataacggtgcagaacgcactcgcgaagttacccgcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatrgaagccatgcgccggaccacgacgtcacgcaggaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggactt

SEQ ID NO: 18 - DNA, 591 bp, Homo sapiens
  allele (326)..(326): rs1042714 (ADRB2); nucleotide indicated by s at position 326 may be either C or G
ctagtctgcgcacataacggtgcagaacgcactcgcgaagcggcttcttcagagcacgggctggaactggcagttacccggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatggaagccatgcgccggaccacgasgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttcca

SEQ ID NO: 19 - protein, 132 aa, Homo sapiens
  MISC_FEATURE (1)..(1): Met at position 1 is removed during processing;
MAFDSTWKVDRSENYDKFMEKMGVNIVKRKLAAHDNLKLTITQEGNKFTVKESSXFRNIEVVFELGVTFNYNLADGTELRGTWSLEGNKLIGKFKRTDNGNELNTVREIIGDELVQTYVYEGVEAKRIFKKD

SEQ ID NO: 20 - protein, 477 aa, Homo sapiens
  VARIANT (12)..(12): PPARG Pro12Ala; Xaa at position 12 may be either Pro or Ala
MTMVDTEMPFWXTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 21 - protein, 408 aa, Homo sapiens
  VARIANT (64)..(64): ADRB3 Trp64Arg; wherein Xaa at position 64 may be either Trp or Arg
MAPWPHENSSLAPWPDLPTLAPNTANTSGLPGVPWEAALAGALLALAVLATVGGNLLVIVAIAXTPRLQTMTNVFVTSLAAADLVMGLLVVPPAATLALTGHWPLGATGCELWTSVDVLCVTASIETLCALAVDRYLAVTNPLRYGALVTKRCARTAVVLVWVVSAAVSFAPIMSQWWRVGADAEAQRCHSNPRCCAFASNMPYVLLSSSVSFYLPLLVMLFVYARVFVVATRQLRLLRGELGRFPPEESPPAPSRSLAPAPVGTCAPPEGVPACGRRPARLLPLREHRALCTLGLIMGTFTLCWLPFFLANVLRALGGPSLVPGPAFLALNWLGYANSAFNPLIYCRSPDFRSAFRRLLCRCGRRLPPEPCAAARPALFPSGVPAARSSPAQPRLCQRLDGASWGVS

SEQ ID NO: 22 - protein, 413 aa, Homo sapiens
  VARIANT (16)..(16): ADRB2 Gly16Arg; wherein Xaa at position 16 may be either Gly or Arg
MGQPGNGSAFLLAPNXSHAPDHDVTQXRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSPGRNCSTNDSLL

(19) United States
(12) Patent Application Publication
(10) Pub. No.: US 2013/0079612 A1
(43) Pub. Date: Mar. 28, 2013

(54) Methods for Creating Recommended Dietary Regime

(71) Applicant: Access Business Group International LLC, Ada, MI (US)

(72) Inventors: Sheri A. Hunt, Manhattan Beach, CA (US); Valiantsina Kazlova, Stanton, CA (US)

(73) Assignee: Access Business Group International LLC, Ada, MI (US)

(21) Appl. No.: 13/622,485

(22) Filed: Sep. 19, 2012

Related U.S. Application Data
(60) Provisional application No. 61/538,220, filed on Sep. 23, 2011.

(51) Int. Cl. A61B 5/0205; A61B 5/145; A61B 5/021; A61B 5/00; A61B 5/107

(52) U.S. Cl. 600/365; 600/300; 600/587; 600/485; 600/483; 600/309

(57) ABSTRACT

The present invention relates to a method of eating a personalized dietary regime that includes receiving personal information relating to the individual; determining the individual's metabolic profile from at least one of a noninvasive or an invasive measurement; and classifying the subject into a nutrition category selected from the group consisting of a low fat diet; a low carbohydrate diet; a high protein diet; or a balanced diet, wherein the invasive measurement does not include genetic testing.

This application claims priority to U.S. Provisional Application No. 61/538,220 filed Sep. 23, 2011, the entire contents of which are incorporated herein by reference.

### BACKGROUND OF THE INVENTION

The present invention relates to methods for creating a personalized dietary regime for a subject based on the subject's metabolic profile. Advantageously, the present method does not require genetic testing and preferably is conducted in the absence of genetic testing.

It is known to conduct genetic testing and, based on the results of such testing, to characterize a subject's metabolic genotype to their response to diet and/or exercise. However, experience has demonstrated that many individuals do not wish to subject themselves to genetic testing and therefore there is a need for an alternative method to characterize a subject's metabolic profile without the need to conduct genetic testing.

### SUMMARY OF THE INVENTION

The present inventors have diligently studied this problem and have now found that based on the measurement of certain biometric markers of a subject, a biometric profile of the subject can be determined. In particular, based on the measurement of certain biometric markers of a subject, the subject's genotype with respect to metabolism and/or weight management can be predicted with an acceptable level of specificity. From the determination of the metabolic profile, the subject can be characterized into a nutrition category. Certain genotypes can be ascertained and in particular in the present invention, three categories of genotypes are identified: (1) responsive to fat restriction, (2) responsive to carbohydrate restriction, and (3) responsive to balance of fat and carbohydrate. Based on the prediction of the genotype, the subject can be classified into an appropriate nutrition category. Suitable nutrition categories include, but are not limited to, a low fat diet; a low carbohydrate diet; a high protein diet; and a calorie restricted diet. With the nutrition category in mind, a personalized dietary regime for the subject can be created that is suitable for weight management, including weight loss.

The biometric markers include one or more relevant noninvasive and/or invasive measurements of a subject. Suitable noninvasive measurements include, but are not limited to, gender, ethnicity, waist girth, systolic blood pressure, and diastolic blood pressure. Suitable invasive measurements include, but are not limited to, LDL cholesterol, HDL cholesterol, triglycerides (mg/dL), and blood glucose level (fasting blood sugar, mM).

The present invention therefore provides for methods and kits for determining a subject's metabolic profile and creating an appropriate therapeutic/dietary regime or lifestyle recommendation for the subject. According to some embodiments, methods are provided for determining a subject's metabolic profile, classifying the subject into one or more nutritional categories to which the subject is likely to be responsive, and communicating to the subject an appropriate therapeutic/dietary regime or lifestyle recommendation for the subject. In this manner, a personalized weight-management program may be provided to the subject based on a subject's metabolic profile. Such a personalized weight-management program will have obvious benefits (e.g., yield better results in terms of weight loss and weight maintenance) over traditional weight-management programs that do not take into account the subject's metabolic profile. Advantageously, the method of determining the subject's metabolic profile is conducted without the need for any genetic information.

It has also been found that from the use of certain combinations of noninvasive measurements alone, or noninvasive measurements in combination with invasive measurements, a subject's genotype with respect to metabolism and/or weight management can be predicted with an acceptable level of specificity. It is also contemplated that invasive measurements alone and combinations of invasive measurements can be used to predict the subject's genotype. Based on the predicted genotype, the subject can be characterized into a nutrition category selected from the group consisting of a low fat diet; a low carbohydrate diet; a high protein diet; and a calorie restricted diet. As a result, the methods of the present invention can be conducted without requiring the need for genetic testing.

The method of the present invention provides a means for establishing a personalized weight loss program that considers a person's metabolic profile in order to improve weight loss and weight maintenance outcomes relative to a similar program not taking into account a person's metabolic profile.

In some aspects of the present invention, the method includes classifying the subject into a nutrition category selected from the group consisting of a low fat diet; a low carbohydrate diet; a high protein diet; and a calorie restricted diet. In some embodiments where the subject has a metabolic profile that is responsive to fat restriction, the subject is classified as being responsive to a low fat diet. In some embodiments where the subject has a metabolic profile responsive to carbohydrate restriction, the subject is classified as being responsive to a low carbohydrate diet. In some embodiments where the subject has a metabolic profile responsive to a balance of fat and carbohydrate, the subject is classified as being responsive to a balanced diet.

In some embodiments, the noninvasive measurement is obtained in the form of a questionnaire. The questionnaire could be provided to the subject over a communications network.

In some embodiments, the invasive measurement is obtained from analysis of a sample from the patient such as from blood or urine.

In some embodiments, once the dietary regime for the subject has been created, the personalized dietary regime is provided to the subject. In some embodiments, once the personalized dietary regime has been provided to the subject, feedback information is received from the subject related to the effects of the personalized dietary regime. In some embodiments, the method further comprises using the feedback information to create an updated personalized dietary regime according to the effects of the personalized dietary regime on the subject.

It is also contemplated that one or more aspects of the medical history of the subject may be provided to a system for assessment of the personalized dietary regime. This may be obtained from the subject's physician or may be inputted by the subject, in which case an interface with the system may be appropriate.

In another aspect of the invention, there is provided a method for creating a personalized dietary regime, comprising: a memory adapted to store at least one of a noninvasive or invasive measurement relating to a subject; a memory adapted to store personal information relating to the subject; a processor adapted to determine at least one of an invasive and/or noninvasive criteria relevant to a metabolic profile, to determine a genotype with respect to metabolism and/or weight management with an acceptable rate of specifity, and to classify the subject into a nutrition category. Based on the nutrition category a personalized dietary regime for the subject can be created.

In another aspect of the invention, there is provided a system for creating a personalized dietary regime that includes a terminal adapted to receive personal information relating to a subject; a data store adapted to store the personal information relating to the subject, the invasive measurement, and/or the noninvasive measurement; and a determination sub-system adapted to determine at least one of an invasive and/or noninvasive criteria relevant to a metabolic profile, to determine a genotype with respect to metabolism and/or weight management with an acceptable rate of specifity, to classify the subject into a nutrition category and to create the personalized dietary regime for the subject.

In another aspect of the invention, there is provided a server for use in a system for creating a personalized dietary regime, comprising: a data store adapted to store personal information relating to a subject; a data store adapted to store at least one of invasive or noninvasive measurements relating to the subject; and a determination processor adapted to determine at least one of an invasive and/or noninvasive criteria relevant to a metabolic profile, to determine a genotype with respect to metabolism and/or weight management with an acceptable rate of specifity, to classify the subject into a nutrition category, and to create the personalized dietary regime for the subject.

The term “noninvasive measurement” as used in the present specification relates to measurements of a subject that does not require an analysis of a subject's fluid. For example, noninvasive measurements include, but are not limited to, gender, ethnicity, waist girth, blood pressure, systolic blood pressure, diastolic blood pressure, eye color, and natural hair color.

The term “invasive measurement” as used in the present specification relates to measurements of a subject's body fluid, including blood, urine, saliva but according to the present invention, excludes gene testing. Such measurements include, but are not limited to, LDL cholesterol, HDL cholesterol, triglycerides, blood glucose, vitamin D level, and calcium level. Invasive measurements also include those results that are a result of testing required to be conducted by an external health professional or facility.

### DETAILED DESCRIPTION OF THE PREFERRED EMBODIMENTS

The methods of the present invention rely at least in part upon the finding that there is an association between certain biometric markers and the subject's genotype with respect to to metabolism and/or weight management as determined by genetic testing. As a result of this association, noninvasive measurements, certain invasive measurements, and/or the combination of certain noninvasive measurements and invasive measurements of a subject are taken and are used in one or more algorithms, described in more detail below, to arrive a prediction of the subject's genotype with respect to metabolism and/or weight management with an acceptable level of specificity. The methods of the present invention can be conducted without genetic testing.

In certain embodiments, the acceptable level of specificity is greater than 0.5, or greater than 0.6, or greater than 0.7, or greater than 0.8, or greater than 0.9. In other embodiments, the acceptable level of specificity is greater than 0.55, or greater than 0.65, or greater than 0.75, or greater than 0.85, or greater than 0.95.

The present invention provides for tests to determine a subject's “metabolic profile”, which involves measuring one or more biometric markers selected from one or more noninvasive measurements and/or one or more invasive measurements. From the measurements, the subject's genotype with respect to metabolism and/or weight management can be predicted. The predicted genotype is then used to classify the subject into a nutrition category selected from the group consisting of a low fat diet; a low carbohydrate diet; a high protein diet; and a calorie restricted diet. Based on the nutrition category, a personalized dietary regime for the subject can be created.

As used in the present specification and claims the term biometric markers refers to one or more noninvasive measurements of a subject, one or more invasive measurements of a subject, or a combination of one or more noninvasive and invasive measurements. It is has been found that the use of certain biometric markers to determine a subject's genotype with respect to metabolism and/or weight management will correlate with the subject's genotype with respect to metabolism and/or weight management as determined by genetic testing.

### Noninvasive Measurements

While there exist a number of noninvasive measurements that could be considered relevant to metabolism and/or weight management, the present inventors have found that the following noninvasive measurements are most relevant for predicting subject's genotype with respect to metabolism and/or weight management: ethnicity, gender, waist girth (weight around the subject's mid-section, i.e., belly fat), and blood pressure, including systolic pressure and diastolic blood pressure, particularly diastolic blood pressure. It has also been found that the combination of gender and waist girth can be used alone, with other noninvasive measurements, or with invasive measurements the results of which can be used to predict the subject's genotype.

While the above noninvasive measurements are preferred, it is contemplated that other noninvasive measurements might be useful in the method of the present invention. Such other noninvasive measurements might include, but are not limited to, eye color and natural hair color.

The noninvasive measurement may be provided by the subject, a health professional or practitioner, or other person with access to the subject. The results of the measurements may be obtained in the form of a questionnaire, which could be provided in an electronic or non-electronic form. For example, a questionnaire may be provided as part of a subject accessible website through the internet, which is well known and need not be explained in further detail. The website may be restricted through the use of passwords, encryption, or other means so that the subject's privacy may be maintained.

### Invasive Measurements

While there exist a number of invasive measurements that could be considered relevant to weight management, the present inventors have found that the following invasive measurements are most relevant for predicting subject's genotype with respect to metabolism and/or weight management: LDL cholesterol, HDL cholesterol, triglycerides, and blood glucose. Other contemplated invasive measurements may include but are not limited to vitamin D and calcium levels. It is believed that these measurements will be readily available as a result of a periodic doctor's examination or are easily obtainable measurements. The subject or health care provider can provide this information for purposes of the present method.

As with the noninvasive measurements, the invasive measurements may be provided in an electronic or non-electronic manner. Thus, for example, using a secured internet accessible website, an authorized user such as the subject or the subject's authorized health care provider, the authorized user can input the results of the invasive measurements.

It is also contemplated that one or more of the above invasive measurements can be combined with one or more of the above noninvasive measurements. In this regard, it has been found that when the noninvasive measurements ethnicity, gender, waist girth, and diastolic blood pressure are combined with the invasive measurements LDL cholesterol, HDL cholesterol, triglycerides, and blood glucose, the subject's genotype can be predicted at a level of specificity of at least 0.75.

In one embodiment, certain noninvasive measurements of a subject are taken and input into an algorithm to predict a subject's genotype. In one aspect, the measurements are input into statistical software such as JMP from SAS, from which the probability of the subject's genotype is outputted. Based on the predicted genotype, the subject can be classified into a nutrition category and a personalized dietary regime can be created.

In another aspect of the present invention, the created dietary regime can be provided to the subject. Further, after the personalized dietary regime is provided to the subject, feedback information can be received from the subject related to the effects of the personalized dietary regime. In some embodiments, the feedback information can be used to create an updated personalized dietary regime.

### Correlation Between Biometric Marker Results and Genetic Testing

A study was conducted of 104 subjects, who were genetically tested to assess their genotype relevant to their metabolism and weight management. The genetic testing was conducted to determine a subject's genotype with respect to polymorphic loci selected from the FABP2 (rs1799883; G/A) locus, PPARG (rs1801282; C/G) locus, ADRB3 (rs4994; CIT) locus, ADRB2 (rs1042713; A/G) locus, or ADRB2 (rs1042714; C/G) locus, wherein the subject's genotype with respect to the loci provides information about the subject's increased susceptibility to adverse weight management issues.

Briefly, the method for identifying a subject's metabolic genotype includes identifying the subject's genotype with respect to one or more (i.e., 2, 3, or 4) of the FABP2 locus, PPARG locus, ADRB3 locus, and/or ADRB2 locus. According to some embodiments, the method for identifying a subject's metabolic genotype includes identifying the subject's genotype with respect to one or more (i.e., 2, 3, 4, or 5) of the FABP2 (rs1799883; G/A) locus, PPARG (rs1801282; C/G) locus, ADRB3 (rs4994; C/T) locus, ADRB2 (rs1042713; A/G) locus, and/or ADRB2 (rs1042714; C/G) locus.

According to some embodiments, the method includes identifying a subject's single polymorphism metabolic genotype and includes identifying the genotype with respect to a metabolic gene allele selected from the group consisting of FABP2 (rs1799883; G/A) locus, PPARG (rs1801282; C/G) locus, ADRB3 (rs4994; C/T) locus, ADRB2 (rs1042713; A/G) locus, and/or ADRB2 (rs1042714; C/G) locus.

According to some embodiments, the method includes identifying a subject's composite metabolic genotype and includes identifying the genotype with respect to at least two metabolic gene alleles selected from the group consisting of FABP2 (rs1799883; G/A) locus, PPARG (rs1801282; C/G) locus, ADRB3 (rs4994; C/T) locus, ADRB2 (rs1042713; A/G) locus, and/or ADRB2 (rs1042714; C/G) locus.

According to some embodiments, the method includes a subject's metabolic genotype and includes identifying the composite polymorphism genotype with respect to at least three metabolic gene alleles selected from the group consisting of FABP2 (rs1799883; G/A) locus, PPARG (rs1801282; C/G) locus, ADRB3 (rs4994; C/T) locus, ADRB2 (rs1042713; A/G) locus, and/or ADRB2 (rs1042714; C/G) locus.

According to some embodiments, the method includes identifying a subject's metabolic genotype and includes identifying the composite polymorphism genotype with respect to at least four metabolic gene alleles selected from the group consisting of FABP2 (rs1799883; G/A) locus, PPARG (rs1801282; C/G) locus, ADRB3 (rs4994; C/T) locus, ADRB2 (rs1042713; A/G) locus, and/or ADRB2 (rs1042714; C/G) locus.

According to some embodiments, the method includes identifying a subject's metabolic genotype and includes identifying the composite polymorphism genotype with respect to each of the metabolic gene alleles FABP2 (rs1799883; G/A) locus, PPARG (rs1801282; C/G) locus, ADRB3 (rs4994; C/T) locus, ADRB2 (rs1042713; A/G) locus, and/or ADRB2 (rs1042714; C/G) locus.

A subject's single polymorphism metabolic genotype and/or composite metabolic genotype results may be classified according to their relationships to weight management risk, including what constitutes a “less responsive” or “more responsive” result from diet and/or exercise interventions, 2) their associated clinical or health-related biomarker outcomes, 3) their relationships to intervention choices for weight management, and 4) prevalence of each genotype. Table 1 and 2 below defines the alleles of certain metabolic genes and explains the increased risk for susceptibility to certain metabolic disorders/parameters.

TABLE 1
Subject Metabolic Gene/Polymorphism

| | | | Pop. |
|---|---|---|---|
| GENE | Locus/SNP | GENOTYPE | Freq* |
| FABP2 | FABP2 (+54) | 1.2 or 2.2 | 48% |
| | Ala54Thr | G/A or A/A | |
| | Ala = G = allele 1 | (54Ala/Thr or 54Thr/Thr) | |
| | Thr = A = allele 2 | 1.1 | 52% |
| | rs1799883 | GIG | |
| | | (54 Ala/Ala) | |
| PPARG | PPARG (+12) | 1.1 | 81% |
| | Pro12Ala | C/C | |
| | Pro = C = allele 1 | (12Pro/Pro) | |
| | Ala = G = allele 2 | 1.2 or 2.2; | 19% |
| | rs1801282 | C/G or G/G | |
| | | (12Pro/Ala or 12Ala/Ala) | |
| ADRB2 | ADRB2 (+27) | 1.2 or 2.2 | 63% |
| | Gln27Glu | C/G or G/C | |
| | Gln = C = allele 1 | (27Gln/Glu or 27Glu/Glu) | |
| | Glu = G = allele 2 | 1.1 | 37% |
| | rs1042714 | C/C | |
| | | (27Gln/Gln) | |
| ADRB2 | ADRB2 (+16) | 1.1 or 1.2 | 86% |
| | Arg16Gly | 1.2 G/G or G/A | |
| | Gly = G = allele 1 | 1.3 (16Gly/Gly or 16Gly/Arg) | |
| | Arg = A = allele 2 | 2.2 | 14% |
| | rs1042713 | A/A | |
| | | (16Arg/Arg) | |
| ADRB3 | ADRB3 (+64) | 1.2 or 2.2 | 16% |
| | Arg64Trp | T/C or C/C | |
| | Trp = T = allele 1 | (64Trp/Arg or 64Arg/Arg) | |
| | Arg = C = allele 2 | 1.1 | 84% |
| | rs4994 | T/T | |
| | | (64Trp/Trp) | |

*Pop. Freq = population frequency, determined for Caucasians using Quebec Family Study (QFS) database

TABLE 2
Subject Susceptibility Chart Based on Metabolic Genotype

| | | Biomarker | |
|---|---|---|---|
| Genotype | Disease Risk | Risk** | Actionable Information*** |
| FABP2 (+54; | Obesity | ↑BMI | Subjects with this genotype have an |
| rs1799883) | Insulin | ↑Body fat | enhanced absorption of dietary fat |
| 1.2 or 2.2 | Resistance | ↑Abd fat | and a slower metabolism, which |
| | Metabolic | ↑TGs | result in a greater propensity for |
| | Syndrome | ↑Insulin | weight gain and a decreased ability to |
| | | ↑BS | lose weight. Clinical studies indicate |
| | | ↑TNFα | subjects with this genotype will |
| | | ↓RMR | improve their risks of elevated |
| | | | triglycerides, insulin and blood sugars |
| | | | by reducing saturated fat and trans |
| | | | fat, and increasing monounsaturated |
| | | | fats while moderating carbohydrate in the |
| | | | diet. |
| FABP2 (+54; | Negative | No | Subjects with this genotype have |
| rs1799883) | | | normal absorption of dietary fat. |
| 1.1 | | | Clinical studies have demonstrated |
| | | | these subjects respond to a low |
| | | | calorie, low fat diet with weight loss; |
| | | | decreased body fat, and lower LDL |
| | | | cholesterol levels. |
| PPARG | Obesity | ↑BMI | PPARG plays a key role in fat cell |
| (+12; | Diabetes | ↑Abd fat | formation and fat metabolism. Clinical |
| rs1801282) | | ↓HDL | studies indicate subjects with this |
| 1.1 | | | genotype have a high risk of weight |
| | | | gain and are less responsive to the |
| | | | effect of a low calorie diet on weight |
| | | | loss. Those with a high total fat and |
| | | | polyunsaturated fat intake tend to |
| | | | have a significantly higher BMI than |
| | | | the alternative genotype. |
| PPARG | Obesity | ↑BMI | Subjects with this variant have |
| (+12; | | | variations in fat cell formation and fat |
| rs1801282) | | | metabolism that increase their |
| 1.2 or 2.2 | | | sensitivity to the effects of changes in |
| | | | diet. These subjects have an easier |
| | | | time losing weight from a low calorie |
| | | | diet; however, they are at risk to |
| | | | regain it. Women are 5 fold more |
| | | | likely than the alternative genotype to |
| | | | be obese if their habitual |
| | | | carbohydrate intake exceeds 49%. |
| | | | Therefore, modulation of |
| | | | carbohydrate intake will be beneficial |
| | | | to these subjects to prevent their risk |
| | | | of obesity. They do have a higher BMI |
| | | | as a result of a high saturated and low |
| | | | monounsaturated fat intake. |
| | | | Therefore, the quality of fat in their |
| | | | diet is also important. |
| ADRB2 | Obesity | ↑BMI | Subjects with this gene variant are |
| (+27; | Diabetes | ↑Abd fat | less able to mobilize their fat stores |
| rs1042714) | | ↑TGs | for energy. Women with this variant |
| 1.2 or 2.2 | | ↑Insulin | have 2½ times the risk of obesity and |
| | | ↑BS | elevated insulin levels if their habitual |
| | | | carbohydrate intake exceeds 49% of |
| | | | total calories when compared to |
| | | | subjects with the alternative |
| | | | genotype. Modulation of |
| | | | carbohydrate intake has been shown |
| | | | to reduce insulin levels and will be |
| | | | beneficial to these subjects to |
| | | | prevent their risk of obesity and |
| | | | elevated triglycerides. Both men and |
| | | | women with this genotype are more |
| | | | resistant to the weight loss effect of a |
| | | | low calorie diet and aerobic exercise. |
| ADRB2 | Negative | No | Subjects with this genotype have a |
| (+27; | | | normal breakdown of fat for energy. |
| rs1042714) | | | Consuming a high intake of dietary |
| 1.1 | | | carbohydrates shows no specific |
| | | | effect on body weight. Men who |
| | | | engage in regular physical activity |
| | | | have a significantly reduced obesity |
| | | | risk. Overall, subjects with this |
| | | | genotype are likely to respond with |
| | | | weight change and improvement in |
| | | | health outcomes from changes in diet |
| | | | and aerobic exercise. |
| ADRB2 | Obesity | ↑BMI | Subjects with this gene variant are |
| (+16; | | ↑Body fat- | less able to mobilize their fat stores |
| rs1042713) | | Men | for energy in response to a |
| 1.1 or 1.2 | | ↓Body fat- | physiologic stress, such as exercise. |
| | | Women | As a result, they mobilize less cellular |
| | | | fat and lose less weight and body fat |
| | | | than expected in response to aerobic |
| | | | exercise. Additionally, they are at |
| | | | greater risk of rebound weight gain. |
| ADRB2 | Negative | No | Subjects with this genotype mobilize |
| (+16; | | | fat from their fat cells for energy |
| rs1042713) | | | effectively as a result of a low calorie |
| 2.2 | | | diet and aerobic exercise for weight |
| | | | loss. They are more likely to lose the |
| | | | body weight and fat and to keep it |
| | | | off. |
| ADRB3 | Obesity | ↑BMI | Subjects with this genotype do not |
| (+64; rs4994) | DM | ↑Abd fat | break down abdominal fat for energy |
| 1.2 or 2.2 | | ↓RMR | in response to a physiologic stress, |
| | | | such as exercise. As a result, they |
| | | | have a slower energy metabolism and |
| | | | are not so responsive to the |
| | | | beneficial effects of aerobic exercise |
| | | | (weight loss, loss of abdominal fat). |
| ADRB3 | Negative | No | Subjects with this genotype have a |
| (+64; rs4994) | | | normal metabolic rate and |
| 1.1 | | | breakdown of abdominal body fat. |
| | | | Studies have shown these subjects |
| | | | experience weight loss by engaging in |
| | | | light to moderate aerobic exercise. |

**BMI = body mass index, TGs = triglycerides, abd fat = abdominal fat, BS = blood sugars, TNFα = tumor necrosis factor alpha, RMR = resting metabolic rate, HDL = high density lipoprotein.
***Metabolism, nutrition and exercise implications.

Table 3 provides the ethnic prevalence for certain metabolic genotypes.

TABLE 3
Prevalence of the Genotype/Risk (‡) Patterns by Ethnicity

| Gene/Genotype Result | Caucasian (QFS) | Black | Hispanic | Japanese | Chinese | Korean |
|---|---|---|---|---|---|---|
| FABP2 | 48% | 35% | 59% | 58% | 54% | 55% |
| rs1799883 | | | | | | |
| 1.2 or 2.2 ‡ | | | | | | |
| FABP2 | 52% | 65% | 41% | 42% | 46% | 45% |
| rs1799883 | | | | | | |
| 1.1 | | | | | | |
| PPARG | 81% | 96% | 82% | 92% | 95% | 90% |
| rs1801282 | | | | | | |
| 1.1 ‡ | | | | | | |
| PPARG | 19% | 4% | 18% | 8% | 5% | 10% |
| rs1801282 | | | | | | |
| 1.2 or 2.2 | | | | | | |
| ADRB2 | 63% | 35% | 59% | 12-18% | 41-59% | 21% |
| rs1042714 | | | | | | |
| 1.2 or 2.2 ‡ | | | | | | |
| ADRB2 | 37% | 65% | 41% | 82-88% | 41-59% | 79% |
| rs1042714 | | | | | | |
| 1.1 | | | | | | |
| ADRB2 | 86% | 74-80% | 70-81% | 71-81% | 63-73% | 61% |
| rs1042713 | | | | | | |
| 1.1 or 1.2 ‡ | | | | | | |
| ADRB2 | 14% | 20-26% | 19-30% | 19-29% | 27-37% | 39% |
| rs1042713 | | | | | | |
| 2.2 | | | | | | |
| ADRB3 | 16% | 19-27% | 20-35% | 33% | 24-32% | 28% |
| rs4994 | | | | | | |
| 1.2 or 2.2 ‡ | | | | | | |
| ADRB3 | 84% | 73-81% | 65-80% | 67% | 68-76% | 72% |
| rs4994 | | | | | | |
| 1.1 | | | | | | |

‡ = Indicates risk genotype(s)

Combinations of these gene variations affect 1) how subjects respond to specific macronutrients in their diet and 2) their different tendencies in energy metabolism that ultimately influence their ability to maintain or lose weight through exercise. A metabolic genotype determination will help healthy subjects identify a genetic risk for adverse weight management issues that have not yet manifested. Knowing gene-related risks early can assist in making personalized health decisions (nutrition, lifestyle) to preserve future health, as well as provide direction on how best to prioritize a subject's focus on nutrition and lifestyle choices to manage optimal body weight and body composition. Information learned from a subject's metabolic genotype may be used to predict a subject's genetic risk for adverse weight management issues.

The method for selecting an appropriate therapeutic/dietary regimen or lifestyle recommendation for a subject includes: determining a subject's genotype with respect to any four of the polymorphic loci selected from the group consisting of the FABP2 (rs1799883; G/A) locus, PPARG (rs1801282; C/G) locus, ADRB3 (rs4994; C/T) locus, ADRB2 (rs1042713; A/G) locus, and ADRB2 (rs1042714; C/G) locus, wherein the subject's genotype with respect to said loci provides information about the subject's increased susceptibility to adverse weight management issues, and allows the selection of a therapeutic/dietary regimen or lifestyle recommendation that is suitable to the subject's susceptibility to adverse weight management issues.

According to some embodiments, the subject with a combined genotype of FABP2 (rs1799883) 1.1, PPARG (rs1801282) 1.1, ADRB2 (rs1042714) 1.1, and ADRB2 (rs1042713) 2.2, and ADRB3 (rs4994) 1.1 is predicted to be responsive to: a low fat or low carbohydrate, calorie-restricted diet; regular exercise; or both.

According to some embodiments, a subject with a combined genotype of one of FABP2 (rs1799883) 1.1 or 1.2 and PPARG (rs1801282) 1.1, and additionally one of ADRB2 (rs1042714) 1.1, 1.2, or 2.2 in combination with ADRB2 (rs1042713) 2.2 and ADRB3 (rs4994) 1.1 is predicted to be responsive to: a low fat, calorie-restricted diet; regular exercise; or both.

According to some embodiments, a subject with a combined genotype of one of PPARG (rs1801282) 1.2 or 2.2 and/or one of ADRB2 (rs1042714) 1.2 or 2.2, in combination with ADRB2 (rs1042713) 2.2 and ADRB3 (rs4994) 1.1 is predicted to be responsive to: a low carbohydrate, calorie-restricted diet; regular exercise; or both.

According to some embodiments, a subject with a combined genotype of one of PPARG (rs1801282) 1.2 or 2.2 and one of FABP2 (rs1799883) 1.1 or 1.2, in combination with ADRB2 (rs1042713) 2.2 and ADRB3 (rs4994) 1.1 is predicted to be responsive to: a low carbohydrate, calorie-restricted diet; regular exercise; or both.

According to some embodiments, a subject with a combined genotype of FABP2 (rs1799883) 1.1 and PPARG (rs1801282) 1.1, in combination with one of ADRB2 (rs1042713) 1.2 or 1.1 or one of ADRB3 (rs4994) 1.2 or 2.2 is predicted to be responsive to a low fat or low carbohydrate, calorie-restricted diet. According to some embodiments, the subject is further predicted to be less responsive to regular exercise.

According to some embodiments, a subject with a combined genotype of one of FABP2 (rs1799883) 1.1 or 1.2 and PPARG (rs1801282) 1.1, in combination with one of ADRB2 (rs1042714) 1.1, 1.2, or 2.2 and either one of ADRB2 (rs1042713) 1.1 or 1.2 or one of ADRB3 (rs4994) 1.2 or 2.2 is predicted to be responsive to: a low fat, calorie-restricted diet. According to some embodiments, the subject is further predicted to be less responsive to regular exercise.

According to some embodiments, a subject with a combined genotype of one of PPARG (rs1801282) 1.2 or 2.2 and/or one of ADRB2 (rs1042714) 1.2 or 2.2, in combination with one of ADRB2 (rs1042713) 1.1 or 1.2 or one of ADRB3 (rs4994) 1.2 or 2.2 is predicted to be responsive to: a low carbohydrate, calorie-restricted diet. According to some embodiments, the subject is further predicted to be less responsive to regular exercise.

According to some embodiments, a subject with a combined genotype of one of PPARG (rs1801282) 1.2 or 2.2 and one of FABP2 (rs1799883) 1.1 or 1.2, in combination with one of ADRB2 (rs1042713) 1.1 or 1.2 or one of ADRB3 (rs4994) 1.2 or 2.2 is predicted to be responsive to: a low carbohydrate, calorie-restricted diet. According to some embodiments, the subject is further predicted to be less responsive to regular exercise.

According to some embodiments, a method is provided for identifying a subject's metabolic genotype comprising: identifying the subject's genotype with respect to at least three of the FABP2 (rs1799883; G/A) locus, PPARG (rs1801282; C/G) locus, ADRB3 (rs4994; C/T) locus, ADRB2 (rs1042713; A/G) locus, and/or ADRB2 (rs1042714; C/G) locus.

According to some embodiments, a method is provided for identifying a subject's metabolic genotype comprising: identifying the subject's genotype with respect to at least four of the FABP2 (rs1799883; G/A) locus, PPARG (rs1801282; C/G) locus, ADRB3 (rs4994; C/T) locus, ADRB2 (rs1042713; A/G) locus, and/or ADRB2 (rs1042714; C/G) locus.

According to some embodiments, methods are provided for selecting an appropriate therapeutic/dietary regimen or lifestyle recommendation for a subject comprising: a) determining a subject's genotype with respect to any four of the polymorphic loci, selected from: FABP2 (rs1799883; G/A) locus; PPARG (rs1801282; C/G) locus; ADRB3 (rs4994; C/T) locus; ADRB2 (rs1042713; A/G) locus; and ADRB2 (rs1042714; C/G) locus; and b) classifying the subject into a nutrition category and/or an exercise category for which the subject is predicted to obtain a likely benefit, wherein the nutrition category is selected from a low fat diet; a low carbohydrate diet; a high protein diet; and a calorie restricted diet, and wherein the exercise category is selected from: light exercise; normal exercise; and vigorous exercise.

According to some embodiments, a method is provided for selecting an appropriate therapeutic/dietary regimen or lifestyle recommendation for a subject comprising: (a) detecting an allelic pattern of at least two alleles selected from the group consisting of FABP2 (rs1799883) allele 1 (Ala or G), FABP2 (rs1799883) allele 2 (Thr or A), PPARG (rs1801282) allele 1 (Pro or C), PPARG (rs1801282) allele 2 (Ala or G), ADRB3 (rs4994) allele 1 (Trp or T), ADRB3 (rs4994) allele 2 (Arg or C), ADRB2 (rs1042713) allele 1 (Gly or G), ADRB2 (rs1042713) allele 2 (Arg or A), ADRB2 (rs1042714) allele 1 (Gln or C) and ADRB2 (rs1042714) allele 2 (Glu or G), wherein the presence of the allelic pattern is predictive of the subject's response to diet and/or exercise and (b) selecting a therapeutic/dietary regimen or lifestyle recommendation that is suitable for the subject's predicted response to diet and/or exercise.

According to some embodiments, a subject with a combined genotype of FABP2 (rs1799883) 1.1 (Ala/Ala or G/G), PPARG (rs1801282) 1.1 (Pro/Pro or C/C), ADRB2 (rs1042714) 1.1 (Gln/Gln or C/C), and ADRB2 (rs1042713) 2.2 (Arg/Arg or A/A), and ADRB3 (rs4994) 1.1 (Trp/Trp or T/T) is predicted to be responsive to: a low fat or low carbohydrate, calorie-restricted diet; regular exercise; or both.

According to some embodiments, a subject with a combined genotype of one of FABP2 (rs1799883) 1.1 (Ala/Ala or G/G) or 1.2 (Ala/Thr or G/A) and PPARG (rs1801282) 1.1 (Pro/Pro or C/C), and additionally one of ADRB2 (rs1042714) 1.1 (Gln/Gln or C/C), 1.2 (Gln/Glu or C/G), or 2.2 (Glu/Glu or G/G) in combination with ADRB2 (rs1042713) 2.2 (Arg/Arg or A/A) and ADRB3 (rs4994) 1.1 (Trp/Trp or T/T) is predicted to be responsive to: a low fat, calorie-restricted diet; regular exercise; or both.

According to some embodiments, a subject with a combined genotype of one of PPARG (rs1801282) 1.2 (Pro/Ala (C/G) or 2.2 (Ala/Ala or G/G) and/or one of ADRB2 (rs1042714) 1.2 (Gln/Glu or C/G) or 2.2 (Glu/Glu or G/G), in combination with ADRB2 (rs1042713) 2.2 (Arg/Arg or A/A) and ADRB3 (rs4994) 1.1 (Trp/Trp or T/T) is predicted to be responsive to: a low carbohydrate, calorie-restricted diet; regular exercise; or both.

According to some embodiments, a subject with a combined genotype of one of PPARG (rs1801282) 1.2 (Pro/Ala or C/G) or 2.2 (Ala/Ala or G/G) and one of FABP2 (rs1799883) 1.1 (Ala/Ala or G/G) or 1.2 (Ala/Thr or G/A), in combination with ADRB2 (rs1042713) 2.2 (Arg/Arg or A/A) and ADRB3 (rs4994) 1.1 (Trp/Trp or T/T) is predicted to be responsive to: a low carbohydrate, calorie-restricted diet; regular exercise; or both.

According to some embodiments, a subject with a combined genotype of FABP2 (rs1799883) 1.1 (Ala/Ala or G/G) and PPARG (rs1801282) 1.1 (Pro/Pro or C/C), in combination with one of ADRB2 (rs1042713) 1.2 (Gly/Arg or G/A) or 2.2 (Arg/Arg or A/A) or one of ADRB3 (rs4994) 1.2 (Arg/Trp or T/C) or 2.2 (Arg/Arg or C/C) is predicted to be responsive to a low fat or low carbohydrate, calorie-restricted diet. According to some embodiments, the subject is further predicted to be less responsive to regular exercise.

According to some embodiments, a subject with a combined genotype of one of FABP2 (rs1799883) 1.1 (Ala/Ala or G/G) or 1.2 (Ala/Thr or G/A) and PPARG (rs1801282) 1.1 (Pro/Pro or C/C), in combination with one of ADRB2 (rs1042714) 1.1 (Gln/Gln or C/C), 1.2 (Gln/Glu or C/G), or 2.2 (Glu/Glu or G/G) and either one of ADRB2 (rs1042713) 1.1 (Gly/Gly or G/G) or 1.2 (Gly/Arg or G/A) or one of ADRB3 (rs4994) 1.2 (Trp/Arg or T/C) or 2.2 (Arg/Arg or C/C) is predicted to be responsive to: a low fat, calorie-restricted diet. According to some embodiments, the subject is further predicted to be less responsive to regular exercise.

According to some embodiments, a subject with a combined genotype of one of PPARG (rs1801282) 1.2 (Pro/Ala or C/G) or 2.2 (Ala/Ala or G/G) and/or one of ADRB2 (rs1042714) 1.2 (Gln/Glu or C/G) or 2.2 (Glu/Glu or G/G), in combination with one of ADRB2 (rs1042713) 1.1 (Gly/Gly or G/G) or 1.2 (Gly/Arg or G/A) or one of ADRB3 (rs4994) 1.2 (Trp/Arg or T/C) or 2.2 (Arg/Arg or C/C) is predicted to be responsive to: a low carbohydrate, calorie-restricted diet. According to some embodiments, the subject is further predicted to be less responsive to regular exercise.

According to some embodiments, a subject with a combined genotype of one of PPARG (rs1801282) 1.2 (Pro/Ala or C/G) or 2.2 (Ala/Ala or G/G) and one of FABP2 (rs1799883) 1.1 (Ala/Ala or G/G) or 1.2 (Ala/Thr or G/A), in combination with one of ADRB2 (rs1042713) 1.1 (Gly/Gly or G/G) or 1.2 (Gly/Arg or G/A) or one of ADRB3 (rs4994) 1.2 (Trp/Arg or T/C) or 2.2 (Arg/Arg or C/C) is predicted to be responsive to: a low carbohydrate, calorie-restricted diet. According to some embodiments, the subject is further predicted to be less responsive to regular exercise.

### Detection of Alleles

Allelic patterns, polymorphism patterns, or haplotype patterns can be identified by detecting any of the component alleles using any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected, for example, from within the metabolic gene loci, either flanking the marker of interest (as required for PCR amplification) or directly overlapping the marker (as in allele specific oligonucleotide (ASO) hybridization). In a particularly preferred embodiment, the sample is hybridized with a set of primers, which hybridize 5′ and 3′ in a sense or antisense sequence to the vascular disease associated allele, and is subjected to a PCR amplification.

An allele may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications.

A general guideline for designing primers for amplification of unique human chromosomal genomic sequences is that they possess a melting temperature of at least about 50° C., wherein an approximate melting temperature can be estimated using the formula Tₘₑₗₜ=[2×(# of A or T)+4×(# of G or C)].

Many methods are available for detecting specific alleles at human polymorphic loci. The preferred method for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in a subject. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. W091/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Publication No. W092/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Publication No. W091/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment, the DNA sample is obtained from a bodily fluid, e.g., blood, obtained by known techniques (e.g. venipuncture) or saliva. Alternatively, nucleic acid tests can be performed on dry samples (e.g. hair or skin). When using RNA or protein, the cells or tissues that may be utilized must express a metabolic gene of interest.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of a metabolic gene or haplotype and having about 5, 10, 20, 25, or 30 nucleotides around the mutation or polymorphic region. In a preferred embodiment of the invention, several probes capable of hybridizing specifically to other allelic variants of key metabolic genes are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of a metabolic gene or haplotype under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of a metabolic gene or haplotype is identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety-of sequencing reactions known in the art can be used to directly sequence the allele. Exemplary sequencing reactions include those based on techniques developed by Maxim and Gilbert ((1977) Proc. Natl. Acad Sci USA 74:560) or Sanger (Sanger et al (1977) Proc. Nat. Acad. Sci USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94116101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl. Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mut Y enzyme of E. coli cleaves A at G/A mismatches and the thymidine DNA glycosylase from HeLa cells cleaves Tat G/T mismatches (Hsu et al. (1994) Carcinogenesis 15:1657-1662). According to an exemplary embodiment, a probe based on an allele of a metabolic gene locus haplotype is hybridized to a CDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify a metabolic gene locus allele. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control metabolif locus alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of alleles in polyacrylamide gels containing a gradient of denaturant is assayed using denaturing gradient gel electrophoresis (DOGE) (Myers et al. (1985) Nature 313:495). When DOGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl. Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of a metabolic gene locus haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In another aspect, the invention features kits for performing the above-described assays. According to some embodiments, the kits of the present invention may include a means for determining a subject's genotype with respect to one or more metabolic gene. The kit may also contain a nucleic acid sample collection means. The kit may also contain a control sample either positive or negative or a standard and/or an algorithmic device for assessing the results and additional reagents and components including: DNA amplification reagents, DNA polymerase, nucleic acid amplification reagents, restrictive enzymes, buffers, a nucleic acid sampling device, DNA purification device, deoxynucleotides, oligonucleotides (e.g. probes and primers) etc.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

As described above, the control may be a positive or negative control. Further, the control sample may contain the positive (or negative) products of the allele detection technique employed. For example, where the allele detection technique is PCR amplification, followed by size fractionation, the control sample may comprise DNA fragments of the appropriate size. Likewise, where the allele detection technique involves detection of a mutated protein, the control sample may comprise a sample of mutated protein. However, it is preferred that the control sample comprises the material to be tested. For example, the controls may be a sample of genomic DNA or a cloned portion of a metabolic gene. Preferably, however, the control sample is a highly purified sample of genomic DNA where the sample to be tested is genomic DNA.

The oligonucleotides present in said kit may be used for amplification of the region of interest or for direct allele specific oligonucleotide (ASO) hybridization to the markers in question. Thus, the oligonucleotides may either flank the marker of interest (as required for PCR amplification) or directly overlap the marker (as in ASO hybridization).

Information obtained using the assays and kits described herein (alone or in conjunction with information on another genetic defect or environmental factor, which contributes to osteoarthritis) is useful for determining whether a non-symptomatic subject has or is likely to develop the particular disease or condition. In addition, the information can allow a more customized approach to preventing the onset or progression of the disease or condition. For example, this information can enable a clinician to more effectively prescribe a therapy that will address the molecular basis of the disease or condition.

The kit may, optionally, also include DNA sampling means. DNA sampling means are well known to one of skill in the art and can include, but not be limited to substrates, such as filter papers, the AmpliCard™ (University of Sheffield, Sheffield, England S10 2JF; Tarlow, J W, et al., J. of Invest. Dermatol. 103:387-389 (1994)) and the like; DNA purification reagents such as Nucleon™ kits, lysis buffers, proteinase solutions and the like; PCR reagents, such as 10× reaction buffers, themostable polymerase, dNTPs, and the like; and allele detection means such as the Hinfl restriction enzyme, allele specific oligonucleotides, degenerate oligonucleotide primers for nested PCR from dried blood.

Another embodiment of the invention is directed to kits for detecting a predisposition for responsiveness to certain diets and/or activity levels. This kit may contain one or more oligonucleotides, including 5′ and 3′ oligonucleotides that hybridize 5′ and 3′ to at least one allele of a metabolic gene locus or haplotype. PCR amplification oligonucleotides should hybridize between 25 and 2500 base pairs apart, preferably between about 100 and about 500 bases apart, in order to produce a PCR product of convenient size for subsequent analysis.

TABLE 5
Particularly preferred primers for use in the diagnostic method of the invention
included are listed.

| | | | | | | PCR |
|---|---|---|---|---|---|---|
| | | | | | | product |
| | | PCR | | | | size |
| Gene | SNP | primer | Position | Sequence | Position | (bp) |
| FABP | rs179988 | FA_F1 | 5′ | TGTTCTTGTGCAAAGGC | 3′ | 311 |
| 2 | 3 | | | AA TGCTACCG | | |
| | | FA_R1 | 5′ | TCTTACCCTGAGTTCAG | 3′ | |
| | | | | TTC CGTCTGC | | |
| ADRB | rs104271 | A1_F1 | 5′ | GCCCCTAGCACCCGACA | 3′ | 422 |
| 2 | 3 | | | AG CTGAGTGT | | |
| | rs104271 | A2_R1 | 5′ | CCAGGCCCATGACCAGA | 3′ | |
| | 4 | | | TC AGCACAG | | |
| ADRB | rs4994 | A3_F2 | 5′ | AAGCGTCGCTACTCCTC | 3′ | 569 |
| 3 | | | | CC CCAAGAGC | | |
| | | A3_R2 | 5′ | GTCACACACAGCACGTC | 3′ | |
| | | | | CA CCGAGGTC | | |
| PPAR | rs180128 | PP_F1 | 5′ | TGCCAGCCAATTCAAGC | 3′ | 367 |
| G | 2 | | | CC AGTCCTTT | | |
| | | PP_R1 | 5′ | ACACAACCTGGAAGACA | 3′ | |
| | | | | A ACTACAAGAGCAA | | |
| | | SBE | | | | |
| Gene | | primer | | Sequence | | |
| FABP | rs179988 | SBE_FA_F1 | 5′ | GAAGGAAATAAATTCAC | 3′ | |
| 2 | 3 | | | A GTCAAAGAATCAAGC | | |
| ADRB | rs104271 | SBE_A1_F2 | 5′ | AACGGCAGCGCCTTCTT | 3′ | |
| 2 | 3 | | | GC TGGCACCCAAT | | |
| | rs104271 | SBE A2 F1 | 5′ | AGCCATGCGCCGGACC | 3′ | |
| | 4 | | | ACG ACGTCACGCAG | | |
| ADRB | rs4994 | SBE_A3_F3 | 5′ | GGGAGGCAACCTGCTG | 3′ | |
| 3 | | | | GTC | | |
| | | | | ATCGTGGCCATCGCC | | |
| PPAR | rs180128 | SBE_PP_R1 | 5′ | GACAGTGTATCAGTGAA | 3′ | |
| G | 2 | | | GG AATCGCTTTCTG | | |

PCR = Polymerase Chain Reaction
SBE = Single Base Extension

The design of additional oligonucleotides for use in the amplification and detection of metabolic gene polymorphic alleles by the method of the invention is facilitated by the availability of both updated sequence information from human chromosome 4q28-q31—which contains the human FABP2 locus, and updated human polymorphism information available for this locus. Suitable primers for the detection of a human polymorphism in metabolic genes can be readily designed using this sequence information and standard techniques known in the art for the design and optimization of primers sequences. Optimal design of such primer sequences can be achieved, for example, by the use of commercially available primer selection programs such as Primer 2.1, Primer 3 or GeneFisher (See also, Nicklin M. H. J., Weith A. Duff G. W., “A Physical Map of the Region Encompassing the Human Interleukin-1α, interleukin-1β, and Interleukin-1 Receptor Antagonist Genes” Genomics 19: 382 (1995); Nothwang H. G., et al. “Molecular Cloning of the Interleukin-1 gene Cluster: Construction of an Integrated YAC/PAC Contig and a partial transcriptional Map in the Region of Chromosome 2q13” Genomics 41: 370 (1997); Clark, et al. (1986) Nucl. Acids. Res., 14:7897-7914 [published erratum appears in Nucleic Acids Res., 15:868 (1987) and the Genome Database (GDB) project).

Further details of the above method are found in published US application 2010/0105038, incorporated herein in its entirety by reference.

Noninvasive and invasive measurements were also taken from the subjects. Specifically, the following noninvasive measurements were taken: ethnicity, gender, waist girth, systolic blood pressure and diastolic blood pressure. The following invasive measurements were also taken: LDL cholesterol, HDL cholesterol, triglycerides, and blood glucose. The results of these measurements were input into the SAS JMP software to arrive at a prediction of a subject's genotype based on the subject's biometric markers, i.e., certain combinations of noninvasive measurements, certain combinations of invasive measurements, and certain combinations of both noninvasive measurements and invasive measurements. The predicted genotype was compared with the genotype determined through genetic testing to elucidate whether there existed any correlation between the predicted genotype and the genotype as determined through genetic testing.

It was found that certain combinations of noninvasive measurements and combinations of noninvasive and invasive measurements correlated with the results of the genetic testing. Accordingly, it was concluded that these combinations could be used to predict a subject's genotype with an acceptable level of specificity from which the subject could be classified into a nutritional category. It is, however, contemplated that invasive measurements and combinations of invasive measurements can be correlated with the results of the above genetic testing.

### Nutrition Categories

In some aspects of the present invention, the method includes classifying the subject into a nutrition category selected from the group consisting of a low fat diet (also abbreviated as “FT”); a low carbohydrate diet (also abbreviated as “CR”); a high protein diet; and a calorie restricted diet (also referred to as balanced or abbreviated as “BB”).

In some embodiments where the subject has a predicted genotype of responsive to fat restriction, the subject is classified as being responsive to a low fat diet. According to some embodiments, the low fat diet of the methods described above provide no more than about 35 percent of total calories from fat. In other embodiments, the low fat diet would provide no more than about 20 percent of total calories from fat.

In some embodiments where the subject has a predicted genotype of responsive to a low carbohydrate diet, the subject is classified as being responsive to a low carbohydrate diet. According to some embodiments, the low carbohydrate diet of the methods described above provide less than about 50 percent of total calories from carbohydrates. In other embodiments, the low carbohydrate diet would provide no more than about 45 percent of total calories from carbohydrates.

In some embodiments where the subject has a predicted genotype of responsive to a balance of fat and carbohydrate diet, the subject is classified as being responsive to a balanced diet. According to some embodiments, the balanced diet of the methods described above restricts total calories to less than 95% of the subject's weight management level. In other embodiments, the balanced diet might contain no more than about 35 percent of total calories from carbohydrates.

Nutrition categories are generally classified on the basis of the amount of macronutrients (i.e., fat, carbohydrates, and protein) recommended for a subject based on that subject's metabolic profile. The primary goal of selecting an appropriate therapeutic/dietary regime for a subject is to pair a subject's metabolic profile with the nutrition category to which that subject is most likely to be responsive. A nutrition category is generally expressed in terms of the relative amounts of macronutrients suggested for a subject's diet or in terms of calories restrictions (e.g., restricting the total number of calories a subject receives and/or restricting the number of calories a subject receives from a particular macronutrient). For example, nutrition categories may include, but are not limited to, 1) low fat, low carbohydrate diets; 2) low fat diets, or 3) low carbohydrate diets.

Alternatively, nutrition categories may be classified on the basis of the restrictiveness of certain macronutrients recommended for a subject based on that subject's metabolic genotype. For example, nutrition categories may be expressed as 1) balanced or calorie restricted diets; 2) fat restrictive diets, or 3) carbohydrate restrictive diets. Subjects with a metabolic profile that is responsive to fat restriction or low fat diet tend to absorb more dietary fat into the body and have a slower metabolism. They have a greater tendency for weight gain. Clinical studies have shown these subjects have an easier time reaching a healthy body weight by decreasing total dietary fat. They may have greater success losing weight by following a reduced fat and/or reduced calorie diet. In addition, they benefit from replacing saturated fats with monounsaturated fats within a reduced calorie diet. Clinical studies have also shown these same dietary modifications improve the body's ability to metabolize sugars and fats.

Subjects with a metabolic profile that is responsive to carbohydrate restriction or low carbohydrate diet tend to be more sensitive to weight gain from excessive carbohydrate intake. They may have greater success losing weight by reducing carbohydrates within a reduced calorie diet. Subjects with this metabolic profile are prone to obesity and have difficulty with blood sugar regulation if their daily carbohydrate intake is high, such as where the daily carbohydrate intake exceeds, for example, about 49% of total calories. Carbohydrate reduction has been shown to optimize blood sugar regulation and reduce risk of further weight gain. If they have high saturated and low monounsaturated fats in their diet, risk for weight gain and elevated blood sugar increases. While limiting total calories, these subjects may benefit from restricting total carbohydrate intake and shifting the fat composition of their diet to monounsaturated fats (e.g., a diet low in saturated fat and low in carbohydrate).

Subjects with a metabolic profile that is responsive to a balance of fat and carbohydrate show no consistent need for a low fat or low carbohydrate diet. For subjects with this metabolic profile who are interested in losing weight, a balanced diet restricted in calories has been found to promote weight loss and a decrease in body fat.

A low fat diet refers to a diet that provides between about 10% to less than about 40% of total calories from fat. According to some embodiments, a low fat diet refers to a diet that provides no more than about 35 percent (e.g., no more than about 19%, 21%, 23%, 22%, 24%, 26%, 28%, 33%, etc.) of total calories from fat. According to some embodiments, a low fat diet refers to a diet that provides no more than about 30% of total calories from fat. According to some embodiments, a low fat diet refers to a diet that provides no more than about 25% of total calories from fat. According to some embodiments, a low fat diet refers to a diet that provides no more than about 20% of total calories from fat. According to some embodiments, a low fat diet refers to a diet that provides no more than about 15% of total calories from fat. According to some embodiments, a low fat diet refers to a diet that provides no more than about 10% of total calories from fat.

According to some embodiments, a low fat diet refers to a diet that is between about 10 grams and about 60 grams of fat per day. According to some embodiments, a low fat diet refers to a diet that is less than about 50 grams (e.g., less than about 10, 25, 35, 45, etc.) grams of fat per day. According to some embodiments, a low fat diet refers to a diet that is less than about 40 grams of fat per day. According to some embodiments, a low fat diet refers to a diet that is less than about 30 grams of fat per day. According to some embodiments, a low fat diet refers to a diet that is less than about 20 grams of fat per day.

Fats contain both saturated and unsaturated (monounsaturated and polyunsaturated) fatty acids. According to some embodiments, reducing saturated fat to less than 10% of calories is a diet low in saturated fat. According to some embodiments, reducing saturated fat to less than 15% of calories is a diet low in saturated fat. According to some embodiments, reducing saturated fat to less than 20% of calories is a diet low in saturated fat.

A low carbohydrate (CHO) diet refers to a diet that provides between about 15% to less than about 50% of total calories from carbohydrates. According to some embodiments, a low carbohydrate (CHO) diet refers to a diet that provides no more than about 50% (e.g., no more than about 15%, 18%, 20%, 25%, 30%, 35%, 40%, 45%, etc.) of total calories from carbohydrates. According to some embodiments, a low carbohydrate diet refers to a diet that provides no more than about 45% of total calories from carbohydrates. According to some embodiments, a low carbohydrate diet refers to a diet that provides no more than about 40% of total calories from carbohydrates. According to some embodiments, a low carbohydrate diet refers to a diet that provides no more than about 35% of total calories from carbohydrates. According to some embodiments, a low carbohydrate diet refers to a diet that provides no more than about 30% of total calories from carbohydrates. According to some embodiments, a low carbohydrate diet refers to a diet that provides no more than about 25% of total calories from carbohydrates. According to some embodiments, a low carbohydrate diet refers to a diet that provides no more than about 18% percent of total calories from carbohydrates.

A low carbohydrate (CHO) diet may refer to a diet that restricts the amount of grams of carbohydrate in a diet such as a diet of from about 20 to about 250 grams of carbohydrates per day. According to some embodiments, a low carbohydrate diet comprises no more than about 220 (e.g., no more than about 40, 70, 90, 110, 130, 180, 210, etc.) grams of carbohydrates per day. According to some embodiments, a low carbohydrate diet comprises no more than about 200 grams of carbohydrates per day. According to some embodiments, a low carbohydrate diet comprises no more than about 180 grams of carbohydrates per day. According to some embodiments, a low carbohydrate diet comprises no more than about 150 grams of carbohydrates per day. According to some embodiments, a low carbohydrate diet comprises no more than about 130 grams of carbohydrates per day. According to some embodiments, a low carbohydrate diet comprises no more than about 100 grams of carbohydrates per day. According to some embodiments, a low carbohydrate diet comprises no more than about 75 grams of carbohydrates per day.

A calorie restricted diet or balanced diet refers to a diet that is restricts total calories consumed to below a subject's weight maintenance level (WML), regardless of any preference for a macronutrient. A balanced diet or calorie restricted diet seeks to reduce the overall caloric intake of a subject by, for example, reducing the total caloric intake of a subject to below that subject's WML without a particular focus on restricting the calories consumed from any particular macronutrient. Thus, according to some embodiments, a balanced diet may be expressed as a percentage of a subject's WML. For example, a balanced diet is a diet that comprises a total caloric intake of between about 50% to about 100% WML. According to some embodiments, a balanced diet is a diet that comprises a total caloric intake of less than 100% (e.g., less than about 99%, 97%, 95%, 90%, 85%, 80%, 75%, 70%, 65%, 60%, and 55%) of WML. Within this framework, a balanced diet achieves a healthy or desired balance of macronutrients in the diet and may be: low fat; low saturated fat; low carbohydrate; low fat and low carbohydrate; or low saturated fat and low carbohydrate. For example, a diet may be a low fat, calorie restricted diet (where low fat has the meaning as provided hereinabove). A diet may be a low carbohydrate, calorie restricted diet (where low carbohydrate has the meaning as provided hereinabove). A diet may be a balanced, calorie restricted diet (e.g., relative portions of macronutrients may vary where the total calories consumed is below the WML). According to some embodiments, a low-carbohydrate diet may include the following relative amounts: carbohydrates: 45%, protein: 20%, and fat: 35%.

According to some embodiments, a low-fat diet may include the following relative amounts: carbohydrates: 65%, protein: 15%, fat: 20%).

According to some embodiments, a balanced diet may include the following relative amounts: carbohydrates: 55%, protein: 20%, fat: 25%.

Other low carbohydrate, low fat, balanced diet and calorie restricted diets are well known in the art and can be recommended to a subject depending on the subject's metabolic profile.

The present invention also contemplates providing a system for creating a personalized dietary regime that includes a terminal adapted to receive personal information relating to a subject; a data store adapted to store the personal information relating to the subject, the invasive measurement, and/or the noninvasive measurement; and a determination sub-system adapted to determine at least one of an invasive and/or noninvasive criteria relevant to a metabolic profile, to determine a genotype with respect to metabolism and/or weight management with an acceptable rate of specifity, to classify the subject into a nutrition category and to create the personalized dietary regime for the subject.

The terminal may be any device suitable for connecting to a computer system or network through the internet or otherwise. For example, the terminal may be a computer with a keyboard connected to a computer network, a personal digital assistant, a phone with internet capability, other devices that are able to connect to the internet wirelessly or otherwise. Such terminals are known and need not be explained in further detail.

In another aspect of the invention, there is provided a server for use in a system for creating a personalized dietary regime, comprising: a data store adapted to store personal information relating to a subject; a data store adapted to store at least one of invasive or noninvasive measurements relating to the subject; and a determination processor adapted to determine at least one of an invasive and/or noninvasive criteria relevant to a metabolic profile, to determine a genotype with respect to metabolism and/or weight management with an acceptable rate of specifity, to classify the subject into a nutrition category, and to create the personalized dietary regime for the subject.

### Example 1

The following example provides an algorithm for the analysis from which the subject's genotype can be predicted using noninvasive measurements. As noted above, the algorithm can be implemented using the JMP software from SAS.

The algorithm was established to determine if there existed a correlation between a subject's predicted genotype selected from one of three responsive to carbohydrate restriction, responsive to a balance of fat and carbohydrate, or responsive to fat restriction and the subject's genotype as predicted using certain biometric markers and the subject's genotype as determined from genetic testing. Noninvasive measurement estimates were derived from a questionnaire completed by the subject.

Because there is a correlation between the predicted genotype and the nutrition category (e.g., a genotype of response to a balance of fat and carbohydrate will lead to a nutrition category of BB), the tables below will simply use the abbreviation for the nutrition category. Tables 1 and 2 below provide the estimates for BB and FT, respectively, from which the logarithmic odds can be calculated.

TABLE 1

| | | Lower | |
|---|---|---|---|
| Term for calculating logarithmic odds of genotype BB | Estimate | Value | Upper Value |
| Intercept | 1657.185 | −1800775 | 1804089 |
| Gender[F] | 4.008211 | 1.643268 | 6.373153 |
| Clinical/Tests − Waist Girth | 0.780654 | 0.481936 | 1.079371 |
| Clinical/Tests − Systolic Blood Pressure | −96.6295 | −103097 | 102903.8 |
| Clinical/Tests − HDL Cholesterol | 68.98043 | −73530.8 | 73668.76 |
| Clinical/Tests − LDL Cholesterol | −0.00719 | −0.0403 | 0.025914 |
| Clinical/Tests − Triglycerides | 9.852795 | −10523.8 | 10543.49 |
| Clinical/Tests − Blood Glucose | 31.25623 | −33358.5 | 33421.02 |
| Gender[F] * (Clinical/Tests − Waist Girth − 34.4101) | −0.43097 | −0.66497 | −0.19697 |
| (Clinical/Tests − Waist Girth − 34.4101) * (Clinical/Tests − HDL | 0.023015 | 0.0094 | 0.036629 |
| Cholesterol − 58.5955) | | | |
| (Clinical/Tests − Waist Girth − 34.4101) * (Clinical/Tests − | 0.002047 | −0.00083 | 0.004926 |
| Triglycerides − 118.944) | | | |
| (Clinical/Tests − Systolic Blood Pressure − 116.371) * (Clinical/Tests − | 0.003621 | −0.00157 | 0.00881 |
| HDL Cholesterol − 58.5955) | | | |
| (Clinical/Tests − Systolic Blood Pressure − 116.371) * (Clinical/Tests − | −0.00201 | −0.00367 | −0.00035 |
| LDL Cholesterol − 107.09) | | | |
| (Clinical/Tests − HDL Cholesterol − 58.5955) * (Clinical/Tests − LDL | 0.011695 | 0.006911 | 0.016479 |
| Cholesterol − 107.09) | | | |
| (Clinical/Tests − HDL Cholesterol − 58.5955) * (Clinical/Tests − | −0.00201 | −0.00317 | −0.00085 |
| Triglycerides − 118.944) | | | |
| (Clinical/Tests − HDL Cholesterol − 58.5955) * (Clinical/Tests − Blood | −0.02544 | −0.0368 | −0.01408 |
| Glucose − 93.6067) | | | |
| Bio Info − Race/Ethnicity[A] | −2836.81 | −3025189 | 3019515 |
| Bio Info − Race/Ethnicity[H] | 1418.381 | −1509758 | 1512594 |
| (Clinical/Tests − Systolic Blood Pressure − 116.371 ) * Bio Info − | −193.529 | −206194 | 205807.3 |
| Race/Ethnicity[A] | | | |
| (Clinical/Tests − Systolic Blood Pressure − 116.371) * Bio Info − | 96.78489 | −102904 | 103097.2 |
| Race/Ethnicity[H] | | | |
| (Clinical/Tests − HDL Cholesterol − 58.5955) * Bio Info − | 137.818 | −147062 | 147337.4 |
| Race/Ethnicity[A] | | | |
| (Clinical/Tests − HDL Cholesterol − 58.5955) * Bio Info − | −69.0259 | −73668.8 | 73530.76 |
| Race/Ethnicity[H] | | | |
| (Clinical/Tests − Triglycerides − 118.944) * Bio Info − Race/Ethnicity[A] | 19.71825 | −21047.6 | 21087 |
| (Clinical/Tests − Triglycerides − 118.944) * Bio Info − Race/Ethnicity[H] | −9.88901 | −10543.5 | 10523.75 |
| (Clinical/Tests − Blood Glucose − 93.6067) * Bio Info − | 62.87992 | −66716.6 | 66842.4 |
| Race/Ethnicity[A] | | | |
| (Clinical/Tests − Blood Glucose − 93.6067) * Bio Info − | −31.2613 | −33421 | 33358.5 |
| Race/Ethnicity[H] | | | |

TABLE 2

| | | Lower | |
|---|---|---|---|
| Term for calculating logarithmic odds of genotype FT | Estimate | Value | Upper Value |
| Intercept | 5.104 | −10.0586 | 20.26662 |
| Gender[F] | 0.075331 | −0.78233 | 0.932993 |
| Clinical/Tests − Waist Girth | 0.406267 | 0.214865 | 0.597668 |
| Clinical/Tests − Systolic Blood Pressure | −0.03011 | −0.1046 | 0.044378 |
| Clinical/Tests − HDL Cholesterol | 0.148224 | 0.034441 | 0.262007 |
| Clinical/Tests − LDL Cholesterol | −0.0074 | −0.03246 | 0.017663 |
| Clinical/Tests − Triglycerides | −0.008 | −0.02621 | 0.010215 |
| Clinical/Tests − Blood Glucose | −0.25203 | −0.39528 | −0.10878 |
| Gender[F] * (Clinical/Tests − Waist Girth − 34.4101) | −0.24428 | −0.38655 | −0.10201 |
| (Clinical/Tests − Waist Girth − 34.4101) * (Clinical/Tests − HDL | 0.01634 | 0.004535 | 0.028145 |
| Cholesterol − 58.5955) | | | |
| (Clinical/Tests − Waist Girth − 34.4101) * (Clinical/Tests − | 0.000281 | −0.00218 | 0.00274 |
| Triglycerides − 118.944) | | | |
| (Clinical/Tests − Systolic Blood Pressure − 116.371) * (Clinical/Tests − | 0.008662 | 0.003823 | 0.013501 |
| HDL Cholesterol − 58.5955) | | | |
| (Clinical/Tests − Systolic Blood Pressure − 116.371) * (Clinical/Tests − | −0.00271 | −0.00397 | −0.00145 |
| LDL Cholesterol − 107.09) | | | |
| (Clinical/Tests − HDL Cholesterol − 58.5955) * (Clinical/Tests − LDL | 0.011306 | 0.006839 | 0.015773 |
| Cholesterol − 107.09) | | | |
| (Clinical/Tests − HDL Cholesterol − 58.5955) * (Clinical/Tests − | −0.00204 | −0.00316 | −0.00092 |
| Triglycerides − 118.944) | | | |
| (Clinical/Tests − HDL Cholesterol − 58.5955) * (Clinical/Tests − Blood | −0.02584 | −0.03635 | −0.01533 |
| Glucose − 93.6067) | | | |
| Bio Info − Race/Ethnicity[A] | −1.16703 | −2.51229 | 0.178243 |
| Bio Info − Race/Ethnicity[H] | −1.30344 | −2.64687 | 0.039989 |
| (Clinical/Tests − Systolic Blood Pressure − 116.371) * Bio Info − | −0.16613 | −0.28257 | −0.04969 |
| Race/Ethnicity[A] | | | |
| (Clinical/Tests − Systolic Blood Pressure − 116.371) * Bio Info − | 0.029646 | −0.07248 | 0.131768 |
| Race/Ethnicity[H] | | | |
| (Clinical/Tests − HDL Cholesterol − 58.5955) * Bio Info − | 0.269401 | 0.098014 | 0.440789 |
| Race/Ethnicity[A] | | | |
| (Clinical/Tests − HDL Cholesterol − 58.5955) * Bio Info − | −0.19942 | −0.34415 | −0.0547 |
| Race/Ethnicity[H] | | | |
| (Clinical/Tests − Triglycerides − 118.944) * Bio Info − Race/Ethnicity[A] | −0.00638 | −0.03185 | 0.019101 |
| (Clinical/Tests − Triglycerides − 118.944) * Bio Info − Race/Ethnicity[H] | −0.01909 | −0.03822 | 4.61e −05 |
| (Clinical/Tests − Blood Glucose − 93.6067) * Bio Info − | 0.239408 | 0.01543 | 0.463386 |
| Race/Ethnicity[A] | | | |
| (Clinical/Tests − Blood Glucose − 93.6067) * Bio Info − | −0.07459 | −0.23319 | 0.08401 |
| Race/Ethnicity[H] | | | |

The logarithmic odds of BB/CR (Lin[BB]) and FT/CR (Lin[FT]) using the above estimate value is calculated as shown below:

$$Lin [ BB ] = - 12.869437734776 + Match ( Gender ) ( \begin{matrix}{}_{}^{“}{F}{}_{}^{”} ⇒ 2.28079365657682 \\ {}_{}^{“}{M}{}_{}^{”} ⇒ - 2.2807936565768 \\ else ⇒ .\end{matrix} ) + 0.17068341225703 * Clinical \text{/} Tests - Waist Girth 0.0583147183364 + * Clinical \text{/} Tests - Diastolic Blood Pressure + Match ( Gender ) ( \begin{matrix}{}_{}^{“}{F}{}_{}^{”} ⇒ \begin{matrix}( Clinical \text{/} Tests - Waist Girth - 34.281914893617 ) * \\ - 0.1495375264953\end{matrix} \\ {}_{}^{“}{M}{}_{}^{”} ⇒ \begin{matrix}( Clinical \text{/} Tests - Waist Girth - 34.281914893617 ) * \\ 0.149537526495326\end{matrix} \\ else ⇒ .\end{matrix} ) + Match ( Bio Info - Race \text{/} Ethnicity ) ( \begin{matrix}{}_{}^{“}{A}{}_{}^{”} ⇒ - 0.2011444150442 \\ {}_{}^{“}{H}{}_{}^{”} ⇒ 0.49425605582988 \\ {}_{}^{“}{W}{}_{}^{”} ⇒ - 0.2931116407856 \\ else ⇒ .\end{matrix} ) ( Clinical \text{/} Tests - Diastolic Blood Pressure - 78.2765957446809 ) + * Match ( Bio Info - Race \text{/} Ethnicity ) ( \begin{matrix}{}_{}^{“}{A}{}_{}^{”} ⇒ - 0.997833781092 \\ {}_{}^{“}{H}{}_{}^{”} ⇒ 0.14615828360456 \\ {}_{}^{“}{W}{}_{}^{”} ⇒ - 0.0463749054954 \\ else ⇒ .\end{matrix} )$$

$$Lin [ FT ] = - 7.1802777860798 + Match ( Gender ) ( \begin{matrix}{}_{}^{“}{F}{}_{}^{”} ⇒ 0.04748261586605 \\ {}_{}^{“}{M}{}_{}^{”} ⇒ - 0.0474826158661 \\ else ⇒ .\end{matrix} ) + - 0.0026169254541 * Clinical \text{/} Tests - Waist Girth 0.09060083556438 + * Clinical \text{/} Tests - Diastolic Blood Pressure + Match ( Gender ) ( \begin{matrix}{}_{}^{“}{F}{}_{}^{”} ⇒ \begin{matrix}( Clinical \text{/} Tests - Waist Girth - 34.281914893617 ) * \\ - 0.0 .534473057863\end{matrix} \\ {}_{}^{“}{M}{}_{}^{”} ⇒ \begin{matrix}( Clinical \text{/} Tests - Waist Girth - 34.281914893617 ) * \\ 0.05344730578628\end{matrix} \\ else ⇒ .\end{matrix} ) + Match ( Bio Info - Race \text{/} Ethnicity ) ( \begin{matrix}{}_{}^{“}{A}{}_{}^{”} ⇒ - 0.017459877453 \\ {}_{}^{“}{H}{}_{}^{”} ⇒ - 0.4101089486778 \\ {}_{}^{“}{W}{}_{}^{”} ⇒ 0.42756882613082 \\ else ⇒ .\end{matrix} ) ( Clinical \text{/} Tests - Diastolic Blood Pressure - 78.2765957446809 ) + * Match ( Bio Info - Race \text{/} Ethnicity ) ( \begin{matrix}{}_{}^{“}{A}{}_{}^{”} ⇒ 0.05491466657698 \\ {}_{}^{“}{H}{}_{}^{”} ⇒ - 0.0192223920127 \\ {}_{}^{“}{W}{}_{}^{”} ⇒ - 0.0356922745643 \\ else ⇒ .\end{matrix} )$$

From the above, the probability (likelihood) of a subject having a genotype from one of BB, CR, or FT can be determined as follows:

Prob[FT]=1/(1+Exp(−Lin[FT])+Exp(Lin[BB]−Lin[FT]))

Prob[BB]=1/(1+Exp(Lin[FT]−Lin[BB])+Exp(−Lin[BB]))

Prob[CR]=1/(1+Exp(Lin[FT])+Exp(Lin[BB]))

From this, whichever probability is the greatest, the particular genotype is predicted.

### Example 2

Three subjects were selected and certain biometric markers were measured. In particular, the following noninvasive measurements were taken: gender, ethnicity, waist girth, and diastolic blood pressure. The logarithmic odds of BB/CR (Lin[BB]) and FT/CR (Lin[FT]) using the above estimate values described above were calculated using the algorithms shown above. From that, the probability of the subject's genotype being either BB, FT, or CR was determined and compared to the genotype as determined by genetic testing. In this instance, each of the predicted genotypes matched the genotype as determined by genetic testing. Table 3 below illustrates the results.

TABLE 3

| | | | | Diastolic | | | | | | Most |
|---|---|---|---|---|---|---|---|---|---|---|
| | Genetic | | Waist | Blood | Lin | Lin | Prob | Prob | Prob | Likely |
| Gender | Genotype | Ethnicity | Girth | Pressure | [BB] | [FT] | [BB] | [FT] | [CR] | Genotype |
| F | FT | A | 31.50 | 88.00 | −0.8358 | 1.4228 | 0.0777 | 0.7432 | 0.1791 | FT |
| F | CR | A | 32.00 | 76.00 | −0.3276 | −0.3514 | 0.2973 | 0.2903 | 0.4125 | CR |
| M | BB | H | 68.00 | 84.00 | 7.7276 | 1.4868 | 0.9976 | 0.0019 | 0.0004 | BB |

### Example 3

The following example provides an algorithm for the analysis from which the subject's genotype can be predicted using a combination of noninvasive and invasive measurements. As noted above, the algorithm can be implemented using the JMP software from SAS.

The algorithm was established to determine if there existed a correlation between a subject's predicted genotype selected from one of three responsive to carbohydrate restriction, responsive to a balance of fat and carbohydrate, or responsive to fat restriction and the subject's genotype as predicted using certain biometric markers and the subject's genotype as determined from genetic testing. Noninvasive measurement estimates were derived from a questionnaire completed by the subject. The following noninvasive measurements were used: ethnicity, gender, waist girth and systolic blood pressure. Invasive measurement estimates were derived from samples obtained from the subject. The following invasive measurements were used: LDL cholesterol, HDL cholesterol, triglycerides and blood glucose.

Because there is a correlation between the predicted genotype and the nutrition category (e.g., a genotype of response to a balance of fat and carbohydrate will lead to a nutrition category of BB), the tables below will simply use the abbreviation for the nutrition category. Tables 4 and 5 below provide the estimates for BB and FT, respectively, from which the logarithmic odds can be calculated.

TABLE 4

| Term for calculating logarithmic odds of genotype BB | Estimate | Lower Value | Upper Value |
|---|---|---|---|
| Intercept | 1657.185 | −1800775 | 1804089 |
| Gender[F] | 4.008211 | 1.643268 | 6.373153 |
| Clinical/Tests − Waist Girth | 0.780654 | 0.481936 | 1.079371 |
| Clinical/Tests − Systolic Blood Pressure | −96.6295 | −103097 | 102903.8 |
| Clinical/Tests − HDL Cholesterol | 68.98043 | −73530.8 | 73668.76 |
| Clinical/Tests − LDL Cholesterol | −0.00719 | −0.0403 | 0.025914 |
| Clinical/Tests − Triglycerides | 9.852795 | −10523.8 | 10543.49 |
| Clinical/Tests − Blood Glucose | 31.25623 | −33358.5 | 33421.02 |
| Gender[F] * (Clinical/Tests − Waist Girth − 34.4101) | −0.43097 | −0.66497 | −0.19697 |
| (Clinical/Tests − Waist Girth − 34.4101) * (Clinical/Tests − HDL | 0.023015 | 0.0094 | 0.036629 |
| Cholesterol − 58.5955) | | | |
| (Clinical/Tests − Waist Girth − 34.4101) * (Clinical/Tests − | 0.002047 | −0.00083 | 0.004926 |
| Triglycerides − 118.944) | | | |
| (Clinical/Tests − Systolic Blood Pressure − 116.371) * (Clinical/Tests − | 0.003621 | −0.00157 | 0.00881 |
| HDL Cholesterol − 58.5955) | | | |
| (Clinical/Tests − Systolic Blood Pressure − 116.371) * (Clinical/Tests − | −0.00201 | −0.00367 | −0.00035 |
| LDL Cholesterol − 107.09) | | | |
| (Clinical/Tests − HDL Cholesterol − 58.5955) * (Clinical/Tests − LDL | 0.011695 | 0.006911 | 0.016479 |
| Cholesterol − 107.09) | | | |
| (Clinical/Tests − HDL Cholesterol − 58.5955) * (Clinical/Tests − | −0.00201 | −0.00317 | −0.00085 |
| Triglycerides − 118.944) | | | |
| (Clinical/Tests − HDL Cholesterol − 58.5955) * (Clinical/Tests − | −0.02544 | −0.0368 | −0.01408 |
| Blood Glucose − 93.6067) | | | |
| Bio Info − Race/Ethnicity[A] | −2836.81 | −3025189 | 3019515 |
| Bio Info − Race/Ethnicity[H] | 1418.381 | −1509758 | 1512594 |
| (Clinical/Tests − Systolic Blood Pressure − 116.371) * Bio Info − | −193.529 | −206194 | 205807.3 |
| Race/Ethnicity[A] | | | |
| (Clinical/Tests − Systolic Blood Pressure − 116.371) * Bio Info − | 96.78489 | −102904 | 103097.2 |
| Race/Ethnicity[H] | | | |
| (Clinical/Tests − HDL Cholesterol − 58.5955) * Bio Info − | 137.818 | −147062 | 147337.4 |
| Race/Ethnicity[A] | | | |
| (Clinical/Tests − HDL Cholesterol − 58.5955) * Bio Info − | −69.0259 | −73668.8 | 73530.76 |
| Race/Ethnicity[H] | | | |
| (Clinical/Tests − Triglycerides − 118.944) * Bio Info − | 19.71825 | −21047.6 | 21087 |
| Race/Ethnicity[A] | | | |
| (Clinical/Tests − Triglycerides − 118.944) * Bio Info − | −9.88901 | −10543.5 | 10523.75 |
| Race/Ethnicity[H] | | | |
| (Clinical/Tests − Blood Glucose − 93.6067) * Bio Info − | 62.87992 | −66716.6 | 66842.4 |
| Race/Ethnicity[A] | | | |
| (Clinical/Tests − Blood Glucose − 93.6067) * Bio Info − | −31.2613 | −33421 | 33358.5 |
| Race/Ethnicity[H] | | | |

TABLE 5

| Term for calculating logarithmic odds of genotype FT | Estimate | Lower Value | Upper Value |
|---|---|---|---|
| Intercept | 5.104 | −10.0586 | 20.26662 |
| Gender[F] | 0.075331 | −0.78233 | 0.932993 |
| Clinical/Tests − Waist Girth | 0.406267 | 0.214865 | 0.597668 |
| Clinical/Tests − Systolic Blood Pressure | −0.03011 | −0.1046 | 0.044378 |
| Clinical/Tests − HDL Cholesterol | 0.148224 | 0.034441 | 0.262007 |
| Clinical/Tests − LDL Cholesterol | −0.0074 | −0.03246 | 0.017663 |
| Clinical/Tests − Triglycerides | −0.008 | −0.02621 | 0.010215 |
| Clinical/Tests − Blood Glucose | −0.25203 | −0.39528 | −0.10878 |
| Gender[F] * (Clinical/Tests − Waist Girth − 34.4101) | −0.24428 | −0.38655 | −0.10201 |
| (Clinical/Tests − Waist Girth − 34.4101) * (Clinical/Tests − HDL | 0.01634 | 0.004535 | 0.028145 |
| Cholesterol − 58.5955) | | | |
| (Clinical/Tests − Waist Girth − 34.4101) * (Clinical/Tests − | 0.000281 | −0.00218 | 0.00274 |
| Triglycerides − 118.944) | | | |
| (Clinical/Tests − Systolic Blood Pressure − 116.371) * (Clinical/Tests − | 0.008662 | 0.003823 | 0.013501 |
| HDL Cholesterol − 58.5955) | | | |
| (Clinical/Tests − Systolic Blood Pressure − 116.371) * (Clinical/Tests − | −0.00271 | −0.00397 | −0.00145 |
| LDL Cholesterol − 107.09) | | | |
| (Clinical/Tests − HDL Cholesterol − 58.5955) * (Clinical/Tests − LDL | 0.011306 | 0.006839 | 0.015773 |
| Cholesterol − 107.09) | | | |
| (Clinical/Tests − HDL Cholesterol − 58.5955) * (Clinical/Tests − | −0.00204 | −0.00316 | −0.00092 |
| Triglycerides − 118.944) | | | |
| (Clinical/Tests − HDL Cholesterol − 58.5955) * (Clinical/Tests − | −0.02584 | −0.03635 | −0.01533 |
| Blood Glucose − 93.6067) | | | |
| Bio Info − Race/Ethnicity[A] | −1.16703 | −2.51229 | 0.178243 |
| Bio Info − Race/Ethnicity[H] | −1.30344 | −2.64687 | 0.039989 |
| (Clinical/Tests − Systolic Blood Pressure − 116.371) * Bio Info − | −0.16613 | −0.28257 | −0.04969 |
| Race/Ethnicity[A] | | | |
| (Clinical/Tests − Systolic Blood Pressure − 116.371) * Bio Info − | 0.029646 | −0.07248 | 0.131768 |
| Race/Ethnicity[H] | | | |
| (Clinical/Tests − HDL Cholesterol − 58.5955) * Bio Info − | 0.269401 | 0.098014 | 0.440789 |
| Race/Ethnicity[A] | | | |
| (Clinical/Tests − HDL Cholesterol − 58.5955) * Bio Info − | −0.19942 | −0.34415 | −0.0547 |
| Race/Ethnicity[H] | | | |
| (Clinical/Tests − Triglycerides − 118.944) * Bio Info − | −0.00638 | −0.03185 | 0.019101 |
| Race/Ethnicity[A] | | | |
| (Clinical/Tests − Triglycerides − 118.944) * Bio Info − | −0.01909 | −0.03822 | 4.61e −05 |
| Race/Ethnicity[H] | | | |
| (Clinical/Tests − Blood Glucose − 93.6067) * Bio Info − | 0.239408 | 0.01543 | 0.463386 |
| Race/Ethnicity[A] | | | |
| (Clinical/Tests − Blood Glucose − 93.6067) * Bio Info − | −0.07459 | −0.23319 | 0.08401 |
| Race/Ethnicity[H] | | | |

The logarithmic odds of BB/CR (Lin[BB]) and FT/CR (Lin[FT]) using the above estimate value is calculated as shown below:

$$Lin [ BB ] = 1657.18545511847 + Match ( : Gender {}_{}^{“}{F}{}_{}^{”} ⇒ 4.0082106444728 , {}_{}^{“}{M}{}_{}^{”} ⇒ - 4.0082106444728 , . ) + 0.780653635197816 * ( “ Clinical \text{/} Tests - Waist Girth ” ) + - 96.6294891708745 * ( “ Clinical \text{/} Tests - Systolic Blood Pressure ” ) + 68.9804328319944 * ( “ Clinical \text{/} Tests - HDL Cholesterol ” ) + - 0.00719235543745936 * ( “ Clinical \text{/} Tests - LDL Cholesterol ” ) + 9.85279524612583 * ( “ Clinical \text{/} Tests - Triglycerides ” ) + 31.2562303506725 * ( “ Clinical \text{/} Tests - Blood Glucose ” ) + Match ( : Gender , {}_{}^{“}{F}{}_{}^{”} ⇒ ( ( “ Clinical \text{/} Tests - Waist Girth ” ) - 34.4101123595506 ) * - 0.430969031561562 , {}_{}^{“}{M}{}_{}^{”} ⇒ ( ( “ Clinical \text{/} Tests - Waist Girth ” ) - 34.4101123595506 ) * 0.430969031561562 , . ) + ( ( “ Clinical \text{/} Tests - Waist Girth ” ) - 34.4101123595506 ) * ( ( “ Clinical \text{/} Tests - HDL Cholesterol ” ) - 58.5955056179775 ) * 0.0230146730468664 + ( ( “ Clinical \text{/} Tests - Waist Girth ” ) - 34.4101123595506 ) * ( ( “ Clinical \text{/} Tests - Triglycerides ” ) - 118.943820224719 ) * 0.00204741704531309 + ( ( “ Clinical \text{/} Tests - Systolic Blood Pressure ” ) - 116.370786516854 ) * ( ( “ Clinical \text{/} Tests - HDL Cholesterol ” ) - 58.5955056179775 ) * 0.00362130444119151 + ( ( “ Clinical \text{/} Tests - Systolic Blood Pressure ” ) - 116.370786516854 ) * ( ( “ Clinical \text{/} Tests - LDL Cholesterol ” ) - 107.089887640449 ) * - 0.00200918535596019 + ( ( “ Clinical \text{/} Tests - HDL Cholesterol ” ) - 58.5955056179775 ) * ( ( “ Clinical \text{/} Tests - LDL Cholesterol ” ) - 107.089887640449 ) * 0.0116949270981976 + ( ( “ Clinical \text{/} Tests - HDL Cholesterol ” ) - 58.5955056179775 ) * ( ( “ Clinical \text{/} Tests - Triglycerides ” ) - 118.943820224719 ) * - 0.00201128988712169 * + ( ( “ Clinical \text{/} Tests - HDL Cholesterol ” ) - 58.5955056179775 ) * ( ( “ Clinical \text{/} Tests - Blood Glucose ” ) - 93.6067415730337 ) * - 0.0254413685179649 + Match ( ( “ Bio Info - Race \text{/} Ethnicity ” ) , {}_{}^{“}{A}{}_{}^{”} ⇒ - 2836.80691395902 , {}_{}^{“}{H}{}_{}^{”} ⇒ 1418.38106669441 , {}_{}^{“}{W}{}_{}^{”} ⇒ 1418.42584726461 , . ) + ( ( “ Clinical \text{/} Tests - Systolic Blood Pressure ” ) - 116.370786516854 ) * Match ( ( “ Bio Info - Race \text{/} Ethnicity ” ) , {}_{}^{“}{A}{}_{}^{”} ⇒ - 193.529470713466 , {}_{}^{“}{H}{}_{}^{”} ⇒ 96.7848922751083 , {}_{}^{“}{W}{}_{}^{”} ⇒ 96.7445784383578 , . ) + ( ( “ Clinical \text{/} Tests - HDL Cholesterol ” ) - 58.5955056179775 ) * Match ( ( “ Bio Info - Race \text{/} Ethnicity ” ) , {}_{}^{“}{A}{}_{}^{”} ⇒ 137.818027511776 , {}_{}^{“}{H}{}_{}^{”} ⇒ - 69.0259035858048 , {}_{}^{“}{W}{}_{}^{”} ⇒ - 68.7921239259716 , . ) + ( ( “ Clinical \text{/} Tests - Triglycerides ” ) - 118.943820224719 ) * Match ( ( “ Bio Info - Race \text{/} Ethnicity ” ) , {}_{}^{“}{A}{}_{}^{”} ⇒ 19.7182509586527 , {}_{}^{“}{H}{}_{}^{”} ⇒ - 9.88901228418387 , {}_{}^{“}{W}{}_{}^{”} ⇒ - 9.82923867446879 , . ) + ( ( “ Clinical \text{/} Tests - Blood Glucose ” ) - 93.6067415730337 ) * Match ( ( “ Bio Info - Race \text{/} Ethnicity ” ) , {}_{}^{“}{A}{}_{}^{”} ⇒ 62.8799241706162 , {}_{}^{“}{H}{}_{}^{”} ⇒ - 31.2612885709512 , {}_{}^{“}{W}{}_{}^{”} ⇒ - 31.6186355996649 , . )$$

$$Lin [ FT ] = 5.10400043931978 + Match ( : Gender , {}_{}^{“}{F}{}_{}^{”} ⇒ 0.0753305232394228 , {}_{}^{“}{M}{}_{}^{”} ⇒ - 0.0753305232394228 , . ) + 0.406266726122459 * ( “ Clinical \text{/} Tests - Waist Girth ” ) + - 0.0301132465836951 * ( “ Clinical \text{/} Tests - Systolic Blood Pressure ” ) + 0.148224315508763 * ( “ Clinical \text{/} Tests - HDL Cholesterol ” ) + - 0.00739675158127971 * ( “ Clinical \text{/} Tests - LDL Cholesterol ” ) + - 0.00799782299877623 * ( “ Clinical \text{/} Tests - Triglycerides ” ) + - 0.252033733906929 * ( “ Clinical \text{/} Tests - Blood Glucose ” ) + Match ( : Gender , {}_{}^{“}{F}{}_{}^{”} ⇒ ( ( “ Clinical \text{/} Tests - Waist Girth ” ) - 34.4101123595506 ) * - 0.244279687463326 , {}_{}^{“}{M}{}_{}^{”} ⇒ ( ( “ Clinical \text{/} Tests - Waist Girth ” ) - 34.4101123595506 ) * 0.244279687463326 , . ) + ( ( “ Clinical \text{/} Tests - Waist Girth ” ) - 34.4101123595506 ) * ( ( “ Clinical \text{/} Tests - HDL Cholesterol ” ) - 58.5955056179775 ) * 0.0163403762615766 + ( ( “ Clinical \text{/} Tests - Waist Girth ” ) - 34.4101123595506 ) * ( ( “ Clinical \text{/} Tests - Triglycerides ” ) - 118.943820224719 ) * 0.000280772246597931 + ( ( “ Clinical \text{/} Tests - Systolic Blood Pressure ” ) - 116.370786516854 ) * ( ( “ Clinical \text{/} Tests - HDL Cholesterol ” ) - 58.5955056179775 ) * 0.00866194507910996 + ( ( “ Clinical \text{/} Tests - Systolic Blood Pressure ” ) - 116.370786516854 ) * ( ( “ Clinical \text{/} Tests - LDL Cholesterol ” ) - 107.089887640449 ) * - 0.0027122801681444 + ( ( “ Clinical \text{/} Tests - HDL Cholesterol ” ) - 58.5955056179775 ) * ( ( “ Clinical \text{/} Tests - LDL Cholesterol ” ) - 107.089887640449 ) * 0.0113058485127103 + ( ( “ Clinical \text{/} Tests - HDL Cholesterol ” ) - 58.5955056179775 ) * ( ( “ Clinical \text{/} Tests - Triglycerides ” ) - 118.943820224719 ) * - 0.00204065348944438 + ( ( “ Clinical \text{/} Tests - HDL Cholesterol ” ) - 58.5955056179775 ) * ( ( “ Clinical \text{/} Tests - Blood Glucose ” ) - 93.6067415730337 ) * - 0.0258415330225515 + Match ( ( “ Bio Info - Race \text{/} Ethnicity ” ) , {}_{}^{“}{A}{}_{}^{”} ⇒ - 1.16702574136552 , {}_{}^{“}{H}{}_{}^{”} ⇒ - 1.30344048644005 , {}_{}^{“}{W}{}_{}^{”} ⇒ 2.47046622780557 , . ) + ( ( “ Clinical \text{/} Tests - Systolic Blood Pressure ” ) - 116.370786516854 ) * Match ( ( “ Bio Info - Race \text{/} Ethnicity ” ) , {}_{}^{“}{A}{}_{}^{”} ⇒ - 0.1661329573720232 , {}_{}^{“}{H}{}_{}^{”} ⇒ 0.0 .29645795457584 , {}_{}^{“}{W}{}_{}^{”} ⇒ 0.136487161914448 , . ) + ( ( “ Clinical \text{/} Tests - HDL Cholesterol ” ) - 58.5955056179775 ) * Match ( ( “ Bio Info - Race \text{/} Ethnicity ” ) , {}_{}^{“}{A}{}_{}^{”} ⇒ 0.269401144324335 , {}_{}^{“}{H}{}_{}^{”} ⇒ - 0.199424526257851 , {}_{}^{“}{W}{}_{}^{”} ⇒ - 0.0699766180664846 , . ) + ( ( “ Clinical \text{/} Tests - Triglycerides ” ) - 118.943820224719 ) * Match ( ( “ Bio Info - Race \text{/} Ethnicity ” ) , {}_{}^{“}{A}{}_{}^{”} ⇒ - 0.00637666652733274 , {}_{}^{“}{H}{}_{}^{”} ⇒ - 0.0190879119758531 , {}_{}^{“}{W}{}_{}^{”} ⇒ 0.0254645785031858 , . ) + ( ( “ Clinical \text{/} Tests - Blood Glucose ” ) - 93.6067415730337 ) * Match ( ( “ Bio Info - Race \text{/} Ethnicity ” ) , {}_{}^{“}{A}{}_{}^{”} ⇒ 0.239407847565301 , {}_{}^{“}{H}{}_{}^{”} ⇒ - 0.0745902508766663 , {}_{}^{“}{W}{}_{}^{”} ⇒ - 0.164817596688635 , . )$$

From the above, the probability (likelihood) of a subject having a genotype from one of BB, CR, or FT can be determined as follows:

Prob[FT]=1/(1+Exp(−Lin[FT])+Exp(Lin[BB]−Lin[FT]))

Prob[BB]=1/(1+Exp(Lin[FT]−Lin[BB])+Exp(−Lin[BB]))

Prob[CR]=1/(1+Exp(Lin[FT])+Exp(Lin[BB]))

From this, whichever probability is the greatest, the particular genotype is predicted.

### Example 4

Three subjects were selected and certain biometric markers were measured. In particular, the following noninvasive measurements were taken: gender, ethnicity, waist girth, and systolic blood pressure and following invasive measurements were taken: HDL cholesterol, LDL cholesterol, triglycerides, and blood glucose. The logarithmic odds of BB/CR (Lin[BB]) and FT/CR (Lin[FT]) using the above estimate values described above were calculated using the algorithms shown above. From that, the probability of the subject's genotype being either BB, FT, or CR was determined and compared to the genotype as determined by genetic testing. In this instance, each of the predicted genotypes matched the genotype as determined by genetic testing. Table 6 below illustrates the results.

TABLE 6

| | | | | | | | | | | | | | | Most |
|---|---|---|---|---|---|---|---|---|---|---|---|---|---|---|
| | Genetic | Race/ | Waist | Systolic | | | | | Lin | Lin | Prob | Prob | Prob | Likely |
| Gender | Genotype | Ethnicity | Girth | BP | HDL | LDL | tgl | glu | [BB] | [FT] | [BB] | [FT] | [CR] | Genotype |
| F | BB | H | 37 | 132 | 40 | 85 | 277 | 81 | 3.2840 | 1.3962 | 0.8411 | 0.1273 | 0.0315 | BB |
| F | FT | H | 27 | 100 | 61 | 116 | 82 | 86 | −2.2532 | 0.5411 | 0.0372 | 0.6085 | 0.3542 | FT |
| M | CR | W | 33 | 118 | 62 | 126 | 130 | 101 | −11.0703 | −2.2997 | 0.0000 | 0.0911 | 0.9088 | CR |

While the invention has been described with reference to particularly preferred embodiments and examples, those skilled in the art will recognize that various modifications may be made to the invention without departing from the spirit and scope thereof.

What is claimed:

1. A method for creating an appropriate dietary regime for weight loss and/or maintenance for a subject comprising: a) determining the subject's metabolic profile from at least one of noninvasive or an invasive measurement; and b) classifying the subject into a nutrition category selected from the group consisting of a low fat diet; a low carbohydrate diet; a high protein diet; and a calorie restricted diet, wherein the invasive measurement does not include genetic testing.

2. The method of claim 1 wherein the subject's metabolic profile is determined from a combination of a noninvasive measurement or an invasive measurement.

3. The method of claim 1 wherein the noninvasive measurement includes at least one of gender, ethnicity, waist girth, systolic blood pressure, and diastolic blood pressure.

4. The method of claim 1 wherein the noninvasive measurement includes at least two of gender, ethnicity, waist girth, systolic blood pressure, and diastolic blood pressure.

5. The method of claim 1 wherein the noninvasive measurement includes at least three of gender, ethnicity, waist girth, systolic blood pressure, and diastolic blood pressure.

6. The method of claim 1 wherein the noninvasive measurement includes each of gender, ethnicity, waist girth, systolic blood pressure, and diastolic blood pressure.

7. The method of claim 1 wherein the noninvasive measurement includes gender and waist girth.

8. The method of claim 1 wherein the invasive measurement includes at least one of LDL cholesterol, HDL cholesterol, triglycerides (mg/dL), and blood glucose level (fasting blood sugar, mM).

9. The method of claim 1 wherein the invasive measurement includes at least two of LDL cholesterol, HDL cholesterol, triglycerides (mg/dL), and blood glucose level (fasting blood sugar, mM).

10. The method of claim 1 wherein the invasive measurement includes at three of LDL cholesterol, HDL cholesterol, triglycerides (mg/dL), and blood glucose level (fasting blood sugar, mM).

11. The method of claim 1 wherein the invasive measurement includes each of LDL cholesterol, HDL cholesterol, triglycerides (mg/dL), and blood glucose level (fasting blood sugar, mM).

12. The method of claim 1, further comprising classifying the subject into a nutrition category selected from the group consisting of a low fat diet; a low carbohydrate diet; a high protein diet; a balanced diet and a calorie restricted diet.

13. The method of claim 1, wherein the noninvasive measurement includes personal information obtained in the form of a questionnaire.

14. The method of claim 13, wherein the questionnaire is provided to the subject over a communications network.

15. The method of claim 1, wherein the invasive measurement is obtained from analysis of a sample from the subject.

16. The method of claim 1, further comprising providing the dietary regime to the subject based on the classification of the subject into the nutrition category.

17. The method of claim 16 wherein subsequent to providing the personalized dietary regime to the subject, feedback information is received from the subject related to an effect of the personalized dietary regime.

18. The method of claim 17, further comprising using the feedback information to determine an updated personalized dietary regime according to the effect of the personalized dietary regime on the subject.
